(19) United States
(12) Patent Application Publication
(10) Pub. No.: US 2024/0200054 A1
(43) Pub. Date: Jun. 20, 2024

(54) METHODS, CELLS & ORGANISMS

(71) Applicant: Kymab Limited, Cambridge (GB)

(72) Inventors: Allan Bradley, Cambridge (GB); Hanif Ali, Cambridge (GB); E-Chiang Lee, Cambridge (GB)

(21) Appl. No.: 18/512,687

(22) Filed: Nov. 17, 2023

Related U.S. Application Data
(63) Continuation of application No. 16/516,996, filed on Jul. 19, 2019, now Pat. No. 11,920,128.
Application No. 16/516,996 is a continuation of application No. 15/094,624, filed on Apr. 8, 2016, now abandoned.
Application No. 15/094,624 is a continuation of application No. 14/490,549, filed on Sep. 18, 2014, now abandoned.

(30) Foreign Application Priority Data
Sep. 18, 2013 (GB) 1316560.0
Dec. 2, 2013 (GB) 1321210.5

(51) Int. Cl. C12N 15/10; A01K 67/0278; C07K 16/00; C12N 5/0781; C12N 15/90

(52) U.S. Cl. CPC C12N 15/102; A01K 67/0278; C07K 16/00; C12N 5/0635; C12N 15/907; A01K 2207/15; A01K 2217/052; A01K 2217/072; A01K 2227/105; A01K 2267/01; C07K 2317/14; C07K 2317/20; C07K 2317/24; C07K 2317/52; C07K 2317/56; C12N 2510/04; C12N 2800/80

(57) ABSTRACT

The invention relates to an approach for introducing one or more desired insertions and/or deletions of known sizes into one or more predefined locations in a nucleic acid (eg, in a cell or organism genome). They developed techniques to do this either in a sequential fashion or by inserting a discrete DNA fragment of defined size into the genome precisely in a predefined location or carrying out a discrete deletion of a defined size at a precise location. The technique is based on the observation that DNA single-stranded breaks are preferentially repaired through the HDR pathway, and this reduces the chances of indels (eg, produced by NHEJ) in the present invention and thus is more efficient than prior art techniques. The invention also provides sequential insertion and/or deletions using single- or double-stranded DNA cutting.

### CROSS-REFERENCE TO RELATED APPLICATIONS

This application is a continuation of U.S. patent application Ser. No. 16/516,996, filed Jul. 19, 2019, which is a continuation of U.S. patent application Ser. No. 15/094,624, filed Apr. 8, 2016, which is a continuation of U.S. patent application Ser. No. 14/490,549, filed Sep. 18, 2014, which claims the benefit of Great Britain Application No. 1321210.5, filed Dec. 2, 2013, and Great Britain Application No. 1316560.0, filed Sep. 18, 2013, the disclosures of which are herein incorporated by reference in their entireties.

### SEQUENCE LISTING

The instant application contains a Sequence Listing which has been submitted electronically in XML format and is hereby incorporated by reference in its entirety. Said XML file, created on Mar. 5, 2024, is named 747760_SA9-613CON4_ST26.xml and is 90,037 bytes in size.

### FIELD OF THE INVENTION

The inventors have devised an approach for introducing one or more desired insertions and/or deletions of known sizes into one or more predefined locations in a nucleic acid (eg, in a cell or organism genome). They developed techniques to do this either in a sequential fashion or by inserting a discrete DNA fragment of defined size into the genome precisely in a predefined location or carrying out a discrete deletion of a defined size at a precise location. The technique is based on the observation that DNA single-stranded breaks are preferentially repaired through the HDR pathway, and this reduces the chances of indels (eg, produced by NHEJ) in the present invention and thus is more efficient than prior art techniques.

The inventors have also devised new techniques termed sequential endonuclease-mediated homology directed recombination (sEHDR) and sequential Cas-mediated homology directed recombination (sCHDR).

### BACKGROUND

Certain bacterial and archaea strains have been shown to contain highly evolved adaptive immune defence systems, CRISPR/Cas systems, which continually undergo reprogramming to direct degradation of complementary sequences present within invading viral or plasmid DNA. Short segments of foreign DNA, called spacers, are incorporated into the genome between CRISPR repeats, and serve as a ‘memory’ of past exposures. CRISPR spacers are then used to recognize and silence exogenous genetic elements in a manner analogous to RNAi in eukaryotic organisms. The clustered regularly interspaced short palindromic repeats (CRISPR) system including the CRISPR associated (Cas) protein has been reconstituted in vitro by a number of research groups allowing for the DNA cleavage of almost any DNA template without the caveat of searching for the right restriction enzyme cutter. The CRISPR/Cas system also offers a blunt end cleavage creating a dsDNA or, using mutated Cas versions, a selective single strand-specific cleavage (see Cong et al, Wang et al & Mali et al cited below).

Through in vitro studies using Streptococcus pyogenes type II CRISPR/Cas system it has been shown that the only components required for efficient CRISPR/Cas-mediated target DNA or genome modification are a Cas nuclease (eg, a Cas9 nuclease), CRISPR RNA (crRNA) and trans-activating crRNA (tracrRNA). The wild-type mechanism of CRISPR/Cas-mediated DNA cleavage occurs via several steps. Transcription of the CRISPR array, containing small fragments (20-30 base-pairs) of the encountered (or target) DNA, into pre-crRNA, which undergoes maturation through the hybridisation with tracrRNA via direct repeats of pre-crRNA. The hybridisation of the pre-crRNA and tracrRNA, known as guide RNA (gRNA or sgRNA), associates with the Cas nuclease forming a ribonucleoprotein complex, which mediates conversion of pre-crRNA into mature crRNA. Mature crRNA:tracrRNA duplex directs Cas9 to the DNA target consisting of the protospacer and the requisite protospacer adjacent motif (CRISPR/cas protospacer-adjacent motif; PAM) via heteroduplex formation between the spacer region of the crRNA and the protospacer DNA on the host genome. The Cas9 nuclease mediates cleavage of the target DNA upstream of PAM to create a double-stranded break within the protospacer or a strand-specific nick using mutated Cas9 nuclease whereby one DNA strand-specific cleavage motif is mutated (For example, Cas9 nickase contains a D10A substitution) (Cong et al).

It is worth noting that different strains of Streptococcus have been isolated which use PAM sequences that are different from that used by Streptococcus pyogenes Cas9. The latter requires a NGG PAM sequence. CRISPR/Cas systems (for example the Csy4 endoribonulcease in Pseudomonas aeroginosa (see Shah et al)) have been described in other prokaryotic species, which recognise a different PAM sequence (eg, CCN, TCN, TTC, AWG, CC, NNAGNN, NGG, NGGNG). It is noteworthy that the Csy4 (also known as Cas6f) has no sequence homology to Cas9 but the DNA cleavage occurs through a similar mechanism involving the assembly of a Cas-protein-crRNA complex that facilitates target DNA recognition leading to specific DNA cleavage (Haurwitz et al).

In vitro-reconstituted type II CRISPR/Cas system has been adapted and applied in a number of different settings. These include creating selective gene disruption in single or multiple genes in ES cells and also single or multiple gene disruption using a one-step approach using zygotes to generate biallelic mutations in mice. The speed, accuracy and the efficiency at which this system could be applied to genome editing in addition to its multiplexing capability makes this system vastly superior to its predecessor genome editing technologies namely zinc finger nucleases (ZFNs), transcription activator-like effector nucleases (TALENs) and engineered homing meganucleases (Gaj et al & Perez-Pinera et al). These have been successfully used in various eukaryotic hosts but they all suffer from important limitations notably off-target mutagenesis leading to nuclease-related toxicity and also the time and cost of developing such engineered proteins. The CRISPR/Cas system on the other hand is a superior genome editing system by which mutations can be introduced with relative ease simply by designing a single guided RNA complementary to the protospacer sequence on the target DNA.

The dsDNA break induced by an endonuclease, such as Cas9, is subsequently repaired through non-homologous end joining mechanism (NHEJ) whereby the subsequent DNA repair at the breakpoint junction is stitched together with different and unpredictable inserted or deletions (indels) of varying size. This is highly undesirable when precise nucleic acid or genome editing is required. However a predefined precise mutation can be generated using homology directed repair (HDR), eg, with the inclusion of a donor oligo or donor DNA fragment. This approach with Cas9 nuclease has been shown to generate precise predefined mutations but the efficiency at which this occurs in both alleles is low and mutation is seen in one of the strands of the dsDNA target (Wang et al).

The CRISPR/Cas system does therefore have some limitations in its current form.

While it may be possible to modify a desired sequence in one strand of dsDNA, the sequence in the other strand is often mutated through undesirable NHEJ.

### SUMMARY OF THE INVENTION

A first configuration of the present invention provides:—

A method of nucleic acid recombination, the method comprising providing dsDNA comprising first and second strands and
(a) using nucleic acid cleavage to create 5′ and 3′ cut ends in the first strand;
(b) using homologous recombination to insert a nucleotide sequence between the ends, thereby producing a modified first strand; thereby producing DNA wherein the first strand has been modified by said recombination but the second strand has not been modified; and
(c) optionally replicating the modified first strand to produce a progeny dsDNA wherein each strand thereof comprises a copy of the inserted nucleotide sequence; and isolating the progeny dsDNA.
   A second configuration of the present invention provides:—

A method of nucleic acid recombination, the method comprising
(a) using nucleic acid cleavage to create 5′ and 3′ cut ends in a single nucleic acid strand;
(b) using homologous recombination to insert a nucleotide sequence between the ends, wherein the insert sequence comprises a regulatory element or encodes all or part of a protein; and
(c) optionally obtaining the nucleic acid strand modified in step (b) or a progeny nucleic strand comprising the inserted nucleotide sequence.

### A Third Configuration of the Present Invention Provides:—

A method of nucleic acid recombination, the method comprising
(a) using nucleic acid cleavage to create first and second breaks in a nucleic acid strand, thereby creating 5′ and 3′ cut ends and a nucleotide sequence between the ends;
(b) using homologous recombination to delete the nucleotide sequence; and
(c) optionally obtaining the nucleic acid strand modified in step (b) or a progeny nucleic strand comprising the deletion.

In aspects of the configurations of the invention there is provided a method of sequential endonuclease-mediated homology directed recombination (sEHDR) comprising carrying out the method of any preceding configuration a first time and carrying out the method of any preceding configuration a second time. In this way, the invention enables serial nucleic acid modifications, e.g., genome modifications, to be carried out, which may comprise precise sequence deletions, insertions or combinations of these two or more times. For example, it is possible to use this aspect of the invention to “walk along” nucleic acids (e.g., chromosomes in cells) to make relatively large and precise nucleotide sequence deletions or insertions. In an embodiment, one or more Cas endonucleases (e.g., a Cas9 and/or Cys4) are used in a method of sequential Cas-mediated homology directed recombination (sCHDR).

In another aspect, the invention can be described according to the numbered sentences below:

1. A method of nucleic acid recombination, the method comprising providing dsDNA comprising first and second strands and
(a) using nucleic acid cleavage to create 5′ and 3′ cut ends in the first strand;
(b) using homologous recombination to insert a nucleotide sequence between the ends, thereby producing a modified first strand; thereby producing DNA wherein the first strand has been modified by said recombination but the second strand has not been modified; and
(c) optionally replicating the modified first strand to produce a progeny dsDNA wherein each strand thereof comprises a copy of the inserted nucleotide sequence; and isolating the progeny dsDNA.

2. A method of nucleic acid recombination, the method comprising
(a) using nucleic acid cleavage to create 5′ and 3′ cut ends in a single nucleic acid strand;
(b) using homologous recombination to insert a nucleotide sequence between the ends, wherein the insert sequence comprises a regulatory element or encodes all or part of a protein; and
(c) optionally obtaining the nucleic acid strand modified in step (b) or a progeny nucleic strand comprising the inserted nucleotide sequence.

3. The method of any preceding sentence, wherein the insert sequence replaces an orthologous or homologous sequence of the strand.

4. The method of any preceding sentence, wherein the insert nucleotide sequence is at least 10 nucleotides long.

5. The method of any preceding sentence, wherein the insert sequence comprises a site specific recombination site.

6. A method of nucleic acid recombination, the method comprising
(a) using nucleic acid cleavage to create first and second breaks in a nucleic acid strand, thereby creating 5′ and 3′ cut ends and a nucleotide sequence between the ends;
(b) using homologous recombination to delete the nucleotide sequence; and
(c) optionally obtaining the nucleic acid strand modified in step (b) or a progeny nucleic strand comprising the deletion.

7. The method of sentence 6, wherein the deleted sequence comprises a regulatory element or encodes all or part of a protein.

8. The method of any preceding sentence, wherein step (c) is performed by isolating a cell comprising the modified first strand, or by obtaining a non-human vertebrate in which the method has been performed or a progeny thereof.

9. The method of any preceding sentence, wherein the nucleic acid strand or the first strand is a DNA strand.

10. The method of any preceding sentence wherein the product of the method comprises a nucleic acid strand comprising a PAM motif 3′ of the insertion or deletion.

11. The method of any preceding sentence, wherein step (b) is performed by carrying out homologous recombination between an incoming nucleic acid comprising first and second homology arms, wherein the homology arms are substantially homologous respectively to a sequence extending 5′ from the 5′ end and a sequence extending 3′ from the 3′ end.

12. The method of sentence 11, wherein step (b) is performed by carrying out homologous recombination between an incoming nucleic acid comprising an insert nucleotide sequence flanked by the first and second homology arms, wherein the insert nucleotide sequence is inserted between the 5′ and 3′ ends.

13. The method of sentence 12, wherein the insert is as recited in any one of sentences 3 to 5 and there is no further sequence between the homology arms.

14. The method of any one of sentences 11 to 13, wherein each homology arm is at least 20 contiguous nucleotides long.

15. The method of any one of sentences 11 to 14, wherein the first and/or second homology arm comprises a PAM motif.

16. The method of any preceding sentence, wherein Cas endonuclease-mediated cleavage is used in step (a); optionally by recognition of a GG or NGG PAM motif.

17. The method of sentence 16, wherein a nickase is used to cut in step (a).

18. The method of any preceding sentence, wherein the method is carried out in a cell, e.g., a eukaryotic cell.

19. The method of sentence 19, wherein the method is carried out in a mammalian cell.

20. The method of sentence 19, wherein the cell is a rodent (e.g., mouse) ES cell or zygote.

21. The method of any preceding sentence, wherein the method is carried out in a non-human mammal, e.g., a mouse or rat or rabbit.

22. The method of any preceding sentence, wherein each cleavage site is flanked by PAM motif (e.g., a NGG or NGGNG sequence, wherein N is any base and G is a guanine).

23. The method of any preceding sentence, wherein the 3′ end is flanked 3′ by a PAM motif.

24. The method of any preceding sentence, wherein step (a) is carried out by cleavage in one single strand of dsDNA.

25. The method of any preceding sentence, wherein step (a) is carried out by combining in a cell the nucleic acid strand, a Cas endonuclease, a crRNA and a tracrRNA (e.g., provided by one or more gRNAs) for targeting the endonuclease to carry out the cleavage, and optionally an insert sequence for homologous recombination with the nucleic acid strand.

26. The method of any preceding sentence, wherein step (b) is performed by carrying out homologous recombination with an incoming nucleic acid comprising first and second homology arms, wherein the homology arms are substantially homologous respectively to a sequence extending 5′ from the 5′ end and a sequence extending 3′ from the 3′ end, wherein the second homology arm comprises a PAM sequence such that homologous recombination between the second homology arm and the sequence extending 3′ from the 3′ end produces a sequence comprising a PAM motif in the product of the method.

27. A method of sequential endonuclease-mediated homology directed recombination (sEHDR) comprising carrying out the method of any preceding sentence (e.g., when according to sentence 1 using a nickase to cut a single strand of dsDNA; or when dependent from sentence 2 or 5 using a nuclease to cut both strands of dsDNA) a first time and a second time, wherein endonuclease-mediated cleavage is used in each step (a); wherein the product of the first time is used for endonuclease-mediated cleavage the second time, whereby either (i) first and second nucleotide sequences are deleted the first time and the second times respectively; (ii) a first nucleotide sequence is deleted the first time and a second nucleotide sequence is inserted the second time; (iii) a first nucleotide sequence is inserted the first time and a second nucleotide sequence is deleted the second time; or (iv) first and second nucleotide sequences are inserted the first and second times respectively; optionally wherein the nucleic acid strand modification the second time is within 20 or less nucleotides of the nucleic acid strand modification the first time.

28. The method of sentence 27, wherein the first time is carried out according to sentence 6, wherein the incoming nucleic acid comprises no sequence between the first and second homology arms, wherein sequence between the 5′ and 3′ ends is deleted by homologous recombination; and/or the second time is carried out according to sentence 6, wherein step (b) is performed by carrying out homologous recombination between an incoming nucleic acid comprising first and second homology arms, wherein the homology arms are substantially homologous respectively to a sequence extending 5′ from the 5′ end and a sequence extending 3′ from the 3′ end, wherein the incoming nucleic acid comprises no sequence between the first and second homology arms such that sequence between the 5′ and 3′ ends is deleted by homologous recombination; optionally wherein the second arm comprises a PAM motif such that the product of the second time comprises a PAM motif for use in a subsequent Cas endonuclease-mediated method according to any one of sentences 1 to 26.

29. The method of sentence 27, wherein the first time is carried out according to sentence 1 or 2, wherein the incoming nucleic acid comprises the insert sequence between the first and second homology arms, wherein the insert sequence is inserted between the 5′ and 3′ ends by homologous recombination; and/or the second time is carried out according to sentence 1 or 2, wherein step (b) is performed by carrying out homologous recombination between an incoming nucleic acid comprising first and second homology arms, wherein the homology arms are substantially homologous respectively to a sequence extending 5′ from the 5′ end and a sequence extending 3′ from the 3′ end, wherein the insert sequence is inserted between the 5′ and 3′ ends by homologous recombination; optionally wherein the second arm comprises a PAM motif such that the product of the second time comprises a PAM motif for use in a subsequent Cas endonuclease-mediated method according to any one of sentences 1 to 26.

30. The method of sentence 27, wherein one of said first and second times is carried out as specified in sentence 28 and the other time is carried out as specified in sentence 29, wherein at least one sequence deletion and at least one sequence insertion is performed.

31. The method of any preceding sentence, wherein step (a) is carried out using Cas endonuclease-mediated cleavage and a gRNA comprising a crRNA and a tracrRNA.

32. The method of sentence 25 or 31, wherein the crRNA has the structure 5′-X-Y-3′, wherein X is an RNA nucleotide sequence (optionally at least 5 nucleotides long) and Y is a crRNA sequence comprising a nucleotide motif that hybridises with a motif comprised by the tracrRNA, wherein X is capable of hybridising with a nucleotide sequence extending 5′ from the desired site of the 5′ cut end.

33. The method of sentence 25, 31 or 32, wherein Y is 5′-N1UUUUAN2N3GCUA-3′ (SEQ ID NO: 3), wherein each of N1-3 is a A, U, C or G and/or the tracrRNA comprises the sequence (in 5′ to 3′ orientation) UAGCM1UUAAAAM2 (SEQ ID NO: 4), wherein M1 is spacer nucleotide sequence and M2 is a nucleotide.

34. A method of producing a cell or a transgenic non-human organism, the method comprising
(a) carrying out the method of any preceding sentence to (i) knock out a target nucleotide sequence in the genome of a first cell and/or (ii) knock in an insert nucleotide sequence into the genome of a first cell, optionally wherein the insert sequence replaces a target sequence in whole or in part at the endogenous location of the target sequence in the genome; wherein the cell or a progeny thereof can develop into a non-human organism or cell; and
(b) developing the cell or progeny into a non-human organism or a non-human cell.

35. The method of sentence 34, wherein the organism or cell is homozygous for the modification (i) and/or (ii).

36. The method of sentence 34 or 35, wherein the cell is an ES cell, iPS cell, totipotent cell or pluripotent cell.

37. The method of any one of sentences 34 to 36, wherein the cell is a rodent (e.g., a mouse or rat) cell.

38. The method of any one of sentences 34 to 37, wherein the target sequence is an endogenous sequence comprising all or part of a regulatory element or encoding all or part of a protein.

39. The method of any one of sentences 34 to 38, wherein the insert sequence is a synthetic sequence; or comprises a sequence encoding all or part of a protein from a species other than the species from which the first cell is derived; or comprises a regulatory element from said first species.

40. The method of sentence 39, wherein the insert sequence encodes all or part of a human protein or a human protein subunit or domain.

41. A cell or a non-human organism whose genome comprises a modification comprising a non-endogenous nucleotide sequence flanked by endogenous nucleotide sequences, wherein the cell or organism is obtainable by the method of any one of sentences 24 to 40 and wherein the non-endogenous sequence is flanked 3′ by a Cas PAM motif; wherein the cell is not comprised by a human; and one, more or all of (a) to (d) applies
(a) the genome is homozygous for the modification; or comprises the modification at one allele and is unmodified by Cas-mediated homologous recombination at the other allele;
(b) the non-endogenous sequence comprises all or part of a regulatory element or encodes all or part of a protein;
(c) the non-endogenous sequence is at least 20 nucleotides long;
(d) the non-endogenous sequence replaces an orthologous or homologous sequence in the genome.

42. The cell or organism of sentence 41, wherein the non-endogenous sequence is a human sequence.

43. The cell or organism of sentence 41 or 42, wherein the PAM motif comprises a sequence selected from CCN, TCN, TTC, AWG, CC, NNAGNN, NGGNG GG, NGG, WGG, CWT, CTT and GAA.

44. The cell or organism of any one of sentences 41 to 43, wherein there is a PAM motif no more than 10 nucleotides (e.g., 3 nucleotides) 3′ of the non-endogenous sequence.

45. The cell or organism of any one of sentences 41 to 44, wherein the PAM motif is recognised by a Streptococcus Cas9.

46. The cell or organism of any one of claims 41 to 45, which is a non-human vertebrate cell or a non-human vertebrate that expresses one or more human antibody heavy chain variable domains (and optionally no heavy chain variable domains of a non-human vertebrate species).

47. The cell or organism of any one of sentences 41 to 46, which is a non-human vertebrate cell or a non-human vertebrate that expresses one or more human antibody kappa light chain variable domains (and optionally no kappa light chain variable domains of a non-human vertebrate species).

48. The cell or organism of any one of sentences 41 to 47, which is a non-human vertebrate cell or a non-human vertebrate that expresses one or more human antibody lambda light chain variable domains (and optionally no kappa light chain variable domains of a non-human vertebrate species).

49. The cell or organism of any one of sentences 46 to 48, wherein the non-endogenous sequence encodes a human Fc receptor protein or subunit or domain thereof (e.g., a human FcRn or FcY receptor protein, subunit or domain).

50. The cell or organism of any one of sentences 41 to 48, wherein the non-endogenous sequence comprises one or more human antibody gene segments, an antibody variable region or an antibody constant region.

51. The cell or organism of any one of sentences 41 to 50, wherein the insert sequence is a human sequence that replaces or supplements an orthologous non-human sequence.

52. A monoclonal or polyclonal antibody prepared by immunisation of a vertebrate (e.g., mouse or rat) according to any one of sentences 41 to 51 with an antigen.

53. A method of isolating an antibody that binds a predetermined antigen, the method comprising
(a) providing a vertebrate (optionally a mouse or rat) according to any one of sentences 41 to 51;
(b) immunising said vertebrate with said antigen;
(c) removing B lymphocytes from the vertebrate and selecting one or more B lymphocytes expressing antibodies that bind to the antigen;
(d) optionally immortalising said selected B lymphocytes or progeny thereof, optionally by producing hybridomas therefrom; and
(e) isolating an antibody (e.g., and IgG-type antibody) expressed by the B lymphocytes.

54. The method of sentence 53, comprising the step of isolating from said B lymphocytes nucleic acid encoding said antibody that binds said antigen; optionally exchanging the heavy chain constant region nucleotide sequence of the antibody with a nucleotide sequence encoding a human or humanised heavy chain constant region and optionally affinity maturing the variable region of said antibody; and optionally inserting said nucleic acid into an expression vector and optionally a host.

55. The method of sentence 53 or 54, further comprising making a mutant or derivative of the antibody produced by the method of sentence 53 or 54.

56. The use of an isolated, monoclonal or polyclonal antibody according to sentence 52, or a mutant or derivative antibody thereof that binds said antigen, in the manufacture of a composition for use as a medicament.

57. The use of an isolated, monoclonal or polyclonal antibody according to sentence 52, or a mutant or derivative antibody thereof that binds said antigen for use in medicine.

58. A nucleotide sequence encoding an antibody of sentence 52, optionally wherein the nucleotide sequence is part of a vector.

59. A pharmaceutical composition comprising the antibody or antibodies of sentence 52 and a diluent, excipient or carrier.

60. An ES cell, a eukaryotic cell, a mammalian cell, a non-human animal or a non-human blastocyst comprising an expressible genomically-integrated nucleotide sequence encoding a Cas endonuclease.

61. The cell, animal or blastocyst of sentence 60, wherein the endonuclease sequence is constitutively expressible.

62. The cell, animal or blastocyst of sentence 60, wherein the endonuclease sequence is inducibly expressible.

63. The cell, animal or blastocyst of sentence 60, 61 or 62, wherein the endonuclease sequence is expressible in a tissue-specific or stage-specific manner in the animal or a progeny thereof, or in a non-human animal that is a progeny of the cell or blastocyst.

64. The cell or animal of sentence 63, wherein the cell is a non-human embryo cell or the animal is a non-human embryo, wherein the endonuclease sequence is expressible or expressed in the cell or embryo.

65. The cell of animal sentence 64, wherein the endonuclease is operatively linked to a promoter selected from the group consisting of an embryo-specific promoter (e.g., a Nanog promoter, a Pou5fl promoter or a SoxB promoter).

66. The cell, animal or blastocyst of any one of sentences 60 to 65, wherein the Cas endonuclease is at a Rosa 26 locus.

67. The cell, animal or blastocyst of any one of sentences 60 to 65, wherein the Cas endonuclease is operably linked to a Rosa 26 promoter.

68. The cell, animal or blastocyst of any one of sentences 60 to 63, w5erein the Cas endonuclease sequence is flanked 5′ and 3′ by transposon elements (e.g., inverted piggyBac terminal elements) or site-specific recombination sites (e.g., loxP and/or a mutant lox, e.g., lox2272 or lox511; or frt).

69. The cell, animal or blastocyst of sentence 68, comprising one or more restriction endonuclease sites between the Cas endonuclease sequence and a transposon element.

70. The cell, animal or blastocyst of any one of sentences 60 to 69 comprising one or more gRNAs.

71. The cell, animal or blastocyst of sentence 68, 69 or 70, wherein the gRNA(s) are flanked 5′ and 3′ by transposon elements (e.g., inverted piggyBac terminal elements) or site-specific recombination sites (e.g., loxP and/or a mutant lox, e.g., lox2272 or lox511; or frt). 72. Use of the cell, animal or blastocyst of any one of sentences 60 to 71 in a method according to any one of sentences 1 to 51.

### BRIEF DESCRIPTION OF THE FIGURES

FIG. 1 depicts a schematic of precise DNA Insertion in a Predefined Location (KI). gRNA designed against a predefined location can induce DNA nick using Cas9 D10A nickase 5′ of the PAM sequence (shown as solid black box). Alternatively, gRNA can be used together with Cas9 wild-type nuclease to induce double-stranded DNA breaks 5′ of the PAM sequence. The addition of a donor oligo or a donor DNA fragment (single or double stranded) with homology around the breakpoint region containing any form of DNA alterations including addition of endogenous or exogenous DNA can be precisely inserted at the breakpoint junction where the DNA is repaired through HDR.

FIG. 2 depicts a schematic of precise DNA Deletion (KO). gRNAs targeting flanking region of interest can induce two DNA nicks using Cas9 D10A nickase in predefine locations containing the desired PAM sequences (shown as solid black box). Alternatively, gRNAs can be used with Cas9 wild-type nuclease to induce two DSB flanking the region of interest. Addition of a donor oligo or a donor DNA fragment (single or double stranded) with homology to region 5′ of PAM 1 and 3′ of PAM 2 sequence will guide DNA repair in a precise manner via HDR. DNA repair via HDR will reduce the risk of indel formation at the breakpoint junctions and avoid DNA repair through NHEJ and in doing so, it will delete out the region flanked by the PAM sequence and carry out DNA repair in a pre-determined and pre-defined manner.

FIG. 3 depicts precise DNA Deletion and Insertion (KO→KI). gRNAs targeting flanking region of interest can induce two DNA nicks using Cas9 D10A nickase in predefine locations containing the desired PAM sequences (shown as solid black box). Alternatively, gRNAs can be used with Cas9 wild-type nuclease to induce two DSB flanking the region of interest. Addition of a donor oligo or a donor DNA fragment (single or double stranded) with homology to region 5′ of PAM 1 and 3′ to PAM 2 with inclusion of additional endogenous or exogenous DNA, will guide DNA repair in a precise manner via HDR with the concomitant deletion of the region flanked by DSB or nick and the insertion of DNA of interest.

FIG. 4 depicts a schematic of recycling PAM For Sequential Genome Editing (Deletions). gRNAs targeting flanking region of interest can induce two DNA nicks using Cas9 D10A nickase in predefine locations containing the desired PAM sequences (shown as solid black box). Alternatively, gRNAs can be used with Cas9 wild-type nuclease to induce two DSB flanking the region of interest. Addition of a donor oligo or a donor DNA fragment (single or double stranded) with homology to region 5′ of PAM 2 and 3′ of PAM 3 will guide DNA repair in a precise manner via HDR and in doing so, it will delete out the region between PAM 2 and PAM 3. This deletion will retain PAM 3 and thus acts as a site for carrying out another round of CRISPR/Cas mediated genome editing. Another PAM site (e.g., PAM 1) can be used in conjunction with PAM 3 sequence to carry out another round of deletion as described above. Using this PAM recycling approach, many rounds of deletions can be performed in a stepwise deletion fashion, where PAM 3 is recycled after each round. This approach can be used also for the stepwise addition of endogenous or exogenous DNA.

FIG. 5 depicts CRISPR/Cas mediated Lox Insertion to facilitate RMCE. gRNAs targeting flanking region of interest can induce two DNA nicks using Cas9 D10A nickase in predefine locations containing the desired PAM sequences (shown as solid black box). Alternatively, gRNAs can be used with Cas9 wild-type nuclease to induce two DSB flanking the region of interest. Addition of two donor oligos or donor DNA fragments (single or double stranded) with homology to regions 5′ and 3′ of each PAM sequence where the donor DNA contains recombinase recognition sequence (RRS) such as loxP and lox5171 will guide DNA repair in a precise manner via HDR with the inclusion of these RRS. The introduced RRS can be used as a landing pad for inserting any DNA of interest with high efficiency and precisely using recombinase mediated cassette exchange (RMCE). The retained PAM 2 site can be recycled for another round of CRISPR/Cas mediated genome editing for deleting or inserting DNA of interest. Note, the inserted DNA of interest could contain selection marker such as PGK-Puro flanked by PiggyBac LTR to allow for the initial selection and upon successful integration into DNA of interest, the selection marker can be removed conveniently by expressing hyperPbase transposase.

FIG. 6 depicts a schematic of genome modification to produce transposon-excisable Cas9 and gRNA.

FIG. 7 depicts a schematic of genome modification to produce transposon-excisable Cas9 and gRNA

### DETAILED DESCRIPTION OF THE INVENTION

The inventors addressed the need for improved nucleic acid modification techniques. An example of a technique for nucleic acid modification is the application of the CRISPR/Cas system. This system has been shown thus far to be the most advanced genome editing system available due, inter alia, to its broad application, the relative speed at which genomes can be edited to create mutations and its ease of use. The inventors, however, believed that this technology can be advanced for even broader applications than are apparent from the state of the art.

The inventors realised that an important aspect to achieve this would be to find a way of improving the fidelity of nucleic acid modifications beyond that contemplated by the CRISPR/Cas methods known in the art.

Additionally, the inventors realised that only modest nucleic acid modifications had been reported to date. It would be desirable to effect relatively large predefined and precise DNA deletions or insertions using the CRISPR/Cas system.

The inventors have devised an approach for introducing one or more desired insertions and/or deletions of known sizes into one or more predefined locations in a nucleic acid (eg, in a cell or organism genome). They developed techniques to do this either in a sequential fashion or by inserting a discrete DNA fragment of defined size into the genome precisely in a predefined location or carrying out a discrete deletion of a defined size at a precise location. The technique is based on the observation that DNA single-stranded breaks are preferentially repaired through the HDR pathway, and this reduces the chances of indels (eg, produced by NHEJ) in the present invention and thus is more efficient than prior art techniques.

To this end, the invention provides:—

A method of nucleic acid recombination, the method comprising providing double stranded DNA (dsDNA) comprising first and second strands and
(a) using nucleic acid cleavage to create 5′ and 3′ cut ends in the first strand; and
(b) using homologous recombination to insert a nucleotide sequence between the ends, thereby producing a modified first strand; thereby producing DNA wherein the first strand has been modified by said recombination but the second strand has not been modified.

Optionally the method further comprises replicating the modified first strand to produce a progeny dsDNA wherein each strand thereof comprises a copy of the insert nucleotide sequence. Optionally the method comprises (c) isolating the progeny dsDNA, eg, by obtaining a cell containing said progeny dsDNA. Replication can be effected, for example in a cell. For example, steps (a) and (b) are carried out in a cell and the cell is replicated, wherein the machinery of the cell replicates the modified first strand, eg, to produce a dsDNA progeny in which each strand comprises the modification.

Optionally, in any configuration, aspect, example or embodiment of the invention, the modified DNA strand resulting from step (b) is isolated.

Optionally, in any configuration, aspect, example or embodiment of the invention, the method is carried out in vitro. For example, the method is carried out in a cell or cell population in vitro.

Alternatively, optionally, in any configuration, aspect, example or embodiment of the invention, the method is carried out to modify the genome of a virus.

Alternatively, optionally, in any configuration, aspect, example or embodiment of the invention, the method is carried out in vivo in an organism. In an example, the organism is a non-human organism. In an example it is a plant or an animal or an insect or a bacterium or a yeast. For example, the method is practised on a vertebrate (eg, a human patient or a non-human vertebrate (e.g., a bird, e.g., a chicken) or non-human mammal such as a mouse, a rat or a rabbit). Optionally, in any configuration, aspect, example or embodiment of the invention, the method is a method of cosmetic treatment of a human or a non-therapeutic, non-surgical, non-diagnostic method, e.g, practised on a human or a non-human vertebrate or mammal (e.g., a mouse or a rat).

The invention also provides:

A method of nucleic acid recombination, the method comprising
(a) using nucleic acid cleavage to create 5′ and 3′ cut ends in a single nucleic acid strand;
(b) using homologous recombination to insert a nucleotide sequence between the ends, wherein the insert sequence comprises a regulatory element or encodes all or part of a protein; and
(c) Optionally obtaining the nucleic acid strand modified in step (b) or a progeny nucleic strand comprising the inserted nucleotide sequence, eg, by obtaining a cell containing said progeny nucleic acid strand.

In an example the progeny strand is a product of the replication of the strand produced by step (b). The progeny strand is, for example, produced by nucleic acid replication in a cell. For example, steps (a) and (b) are carried out in a cell and the cell is replicated, wherein the machinery of the cell replicates the modified strand produced in step (b), e.g, to produce a dsDNA progeny in which each strand comprises the modification.

In an example, the single nucleic acid strand is a DNA or RNA strand.

In an example, the regulatory element is a promoter or enhancer.

Optionally, in any configuration, aspect, example or embodiment of the invention, the inserted nucleotide sequence is a plant, animal, vertebrate or mammalian sequence, e.g., a human sequence. For example, the sequence encodes a complete protein, polypeptide, peptide, domain or a plurality of any one of these. In an example, the inserted sequence confers a resistance property to a cell comprising the modified nucleic acid produced by the method of the invention (e.g., herbicide, viral or bacterial resistance). In an example, the inserted sequence encodes an interleukin, receptor (e.g., a cell surface receptor), growth factor, hormone, antibody (or variable domain or binding site thereof), antagonist, agonist; eg, a human version of any of these. In an example, the inserted sequence is an exon.

Optionally, in any configuration, aspect, example or embodiment of the invention, the inserted nucleotide sequence replaces an orthologous or homologous sequence of the strand (e.g, the insert is a human sequence that replaces a plant, human or mouse sequence). For example, the method is carried out in a mouse or mouse cell and the insert replaces an orthologous or homologous mouse sequence (e.g., a mouse biological target protein implicated in disease). For example, the method is carried out (e.g., in vitro) in a human cell and the insert replaces an orthologous or homologous human sequence (e.g., a human biological target protein implicated in disease, e.g., a mutated form of a sequence is replaced with a different (e.g., wild-type) human sequence, which may be useful for correcting a gene defect in the cell. In this embodiment, the cell may be a human ES or iPS or totipotent or pluripotent stem cell and may be subsequently introduced into a human patient in a method of gene therapy to treat and/or prevent a medical disease or condition in the patient).

Optionally, in any configuration, aspect, example or embodiment of the invention, the inserted nucleotide sequence is at least 10 nucleotides long, eg, at least 20, 30, 40, 50, 60, 70, 80, 90, 100, 150, 200, 300, 400, 500, 600, 700, 800 or 900 nucleotides, or at least 1, 2, 3, 5, 10, 20, 50 or 100 kb long.

Optionally, in any configuration, aspect, example or embodiment of the invention, the insert sequence comprises a site specific recombination site, eg, a lox, frt or rox site. For example, the site can be a loxP, lox511 or lox2272 site.

The invention also provides:—

A method of nucleic acid recombination, the method comprising
(a) using nucleic acid cleavage to create first and second breaks in a nucleic acid strand, thereby creating 5′ and 3′ cut ends and a nucleotide sequence between the ends;
(b) using homologous recombination to delete the nucleotide sequence; and
(c) optionally obtaining the nucleic acid strand modified in step (b) or a progeny nucleic strand comprising the deletion.

In an example the progeny strand is a product of the replication of the strand produced by step (b). The progeny strand is, for example, produced by nucleic acid replication in a cell. For example, steps (a) and (b) are carried out in a cell and the cell is replicated, wherein the machinery of the cell replicates the modified strand produced in step (b), eg, to produce a dsDNA progeny in which each strand comprises the modification.

In an example, the single nucleic acid strand is a DNA or RNA strand.

In an example, the deleted sequence comprises a regulatory element or encodes all or part of a protein. In an embodiment, the deleted regulatory element is a promoter or enhancer.

Optionally, in any configuration, aspect, example or embodiment of the invention, the deleted nucleotide sequence is a plant, animal, vertebrate or mammalian sequence, e.g., a human sequence. For example, the sequence encodes a complete protein, polypeptide, peptide, domain or a plurality of any one of these. In an example, the deleted sequence encodes an interleukin, receptor (e.g., a cell surface receptor), growth factor, hormone, antibody (or variable domain or binding site thereof), antagonist, agonist; e.g., a non-human version of any of these. In an example, the deleted sequence is an exon.

Optionally, in any configuration, aspect, example or embodiment of the invention, the deleted nucleotide sequence is replaced by an orthologous or homologous sequence of a different species or strain (e.g., a human sequence replaces an orthologous or homologous plant, human or mouse sequence). For example, the method is carried out in a mouse or mouse cell and the insert replaces an orthologous or homologous mouse sequence (e.g., a mouse biological target protein implicated in disease). For example, the method is carried out (e.g., in vitro) in a human cell and the insert replaces an orthologous or homologous human sequence (e.g., a human biological target protein implicated in disease, e.g., a mutated form of a sequence is replaced with a different (e.g., wild-type) human sequence, which may be useful for correcting a gene defect in the cell. In this embodiment, the cell may be a human ES or iPS or totipotent or pluripotent stem cell and may be subsequently introduced into a human patient in a method of gene therapy to treat and/or prevent a medical disease or condition in the patient).

Optionally, in any configuration, aspect, example or embodiment of the invention, the deleted nucleotide sequence is at least 10 nucleotides long, eg, at least 20, 30, 40, 50, 60, 70, 80, 90, 100, 150, 200, 300, 400, 500, 600, 700, 800 or 900 nucleotides, or at least 1, 2, 3, 5, 10, 20, 50 or 100 kb long.

Optionally, in any configuration, aspect, example or embodiment of the invention, step (c) is performed by isolating a cell comprising the modified first strand, or by obtaining a non-human vertebrate in which the method has been performed or a progeny thereof.

Optionally, in any configuration, aspect, example or embodiment of the invention, the product of the method comprises a nucleic acid strand comprising a PAM motif 3′ of the insertion or deletion. In an example, the PAM motif is within 10, 9, 8, 7 6, 5, 4 or 3 nucleotides of the insertion or deletion. This is useful to enable serial insertions and/or deletions according to the method as explained further below.

Optionally, in any configuration, aspect, example or embodiment of the invention, the product of the method comprises a nucleic acid strand comprising a PAM motif 5′ of the insertion or deletion. In an example, the PAM motif is within 10, 9, 8, 7 6, 5, 4 or 3 nucleotides of the insertion or deletion. This is useful to enable serial insertions and/or deletions according to the method as explained further below.

Optionally, in any configuration, aspect, example or embodiment of the invention, step (b) is performed by carrying out homologous recombination between an incoming nucleic acid comprising first and second homology arms, wherein the homology arms are substantially homologous respectively to a sequence extending 5′ from the 5′ end and a sequence extending 3′ from the 3′ end. The skilled person will be familiar with constructing vectors and DNA molecules for use in homologous recombination, including considerations such as homology arm size and sequence and the inclusion of selection markers between the arms. For example, the incoming nucleic acid comprises first and second homology arms, and the insert sequence and an optional selection marker sequence (e.g., neo nucleotide sequence). The arms may be at least 20, 30, 40, 50, 100 or 150 nucleotides in length, for example. Where deletion is required, the insert is omitted (although an optional selection marker sequence may or may not be included between the arms).

Thus, in an embodiment of the invention, step (b) is performed by carrying out homologous recombination between an incoming nucleic acid comprising an insert nucleotide sequence flanked by the first and second homology arms, wherein the insert nucleotide sequence is inserted between the 5′ and 3′ ends.

In another embodiment of the invention, the insert is between the homology arms and there is no further sequence between the arms.

In an example, each homology arm is at least 20, 30, 40, 50, 100 or 150 nucleotides long.

Optionally, in any configuration, aspect, example or embodiment of the invention, step (a) is carried out using an endonuclease, eg, a nickase. Nickases cut in a single strand of dsDNA only. For example, the endonuclease is an endonuclease of a CRISPR/Cas system, eg, a Cas9 or Cys4 endnonuclease (e.g., a Cas9 or Cys4 nickase). In an example, the endounuclease recognises a PAM listed in Table 1 below, for example, the endonuclease is a Cas endonuclease that recognises a PAM selected from CCN, TCN, TTC, AWG, CC, NNAGNN, NGGNG GG, NGG, WGG, CWT, CTT and GAA. In an example, the Cas endonuclease is a S pyogenes endonuclease, e.g., a S pyogenes Cas9 endonuclease. In an example, a S. pyogenes PAM sequence or Streptococcus thermophilus LMD-9 PAM sequence is used.

In an example, the endonuclease is a Group 1 Cas endonuclease. In an example, the endonuclease is a Group 2 Cas endonuclease. In an example, the endonuclease is a Group 3 Cas endonuclease. In an example, the endonuclease is a Group 4 Cas endonuclease. In an example, the endonuclease is a Group 7 Cas endonuclease. In an example, the endonuclease is a Group 10 Cas endonuclease.

In an example, the endonuclease recognises a CRISPR/Cas Group 1 PAM. In an example, the endonuclease recognises a CRISPR/Cas Group 2 PAM. In an example, the endonuclease recognises a CRISPR/Cas Group 3 PAM. In an example, the endonuclease recognises a CRISPR/Cas Group 4 PAM. In an example, the endonuclease recognises a CRISPR/Cas Group 7 PAM. In an example, the endonuclease recognises a CRISPR/Cas Group 10 PAM.

In an example, Cas endonuclease-mediated cleavage is used in step (a); optionally by recognition of a GG or NGG PAM motif.

In an example, the first and/or second homology arm comprises a PAM motif. This is useful to enable serial insertions and/or deletions according to the method as explained further below.

An example of a suitable nickase is S pyogenes Cas9 D10A nickase (see Cong et al and the Examples section below).

Optionally, in any configuration, aspect, example or embodiment of the invention, steps (a) and (b) of the method is carried out in a cell, eg a bacterial, yeast, eukaryotic cell, plant, animal, mammal, vertebrate, non-human animal, rodent, rat, mouse, rabbit, fish, bird or chicken cell. For example, the cell is an E coli cell or CHO or HEK293 or Picchia or Saccharomyces cell. In an example, the cell is a human cell in vitro. In one embodiment, the cell is an embryonic stem cell (ES cell, e.g., a human or non-human ES cell) or an induced pluripotent stem cell (iPS cell; e.g., a human, rodent, rat or mouse iPS cell) or a pluripotent or totipotent cell. Optionally the cell is not an embryonic cell, e.g., wherein the cell is not a human embryonic cell. Optionally the cell is not a pluripotent or totipotent cell. In an example, the method is used to produce a human stem cell for human therapy (e.g., an iPS cell generated from a cell of a patient for reintroduction into the patient after the method of the invention has been performed on the cell), wherein the stem cell comprises a nucleotide sequence or gene sequence inserted by the method of the invention. The features of the examples in this paragraph can be combined.

In an example, the method is carried out in a mammalian cell. For example, the cell is a human cell in vitro or a non-human mammalian cell. For example, a non-human (e.g., rodent, rat or mouse) zygote. For example, a single-cell non-human zygote.

In an example, the method is carried out in a plant or non-human mammal, e.g. a rodent, mouse or rat or rabbit, or a tissue or organ thereof (eg, in vitro).

In an example, the 3′ or each cleavage site is flanked 3′ by PAM motif (eg, a motif disclosed herein, such as NGG or NGGNG sequence, wherein N is any base and G is a guanine). For example, one or more or all cleavage sites are flanked 3′ by the sequence 5′-TGGTG-3′. Unlike dsDNA, the PAM is not absolutely required for ssDNA binding and cleavage: A single-stranded oligodeoxynucleotide containing a protospacer with or without a PAM sequence is bound nearly as well as dsDNA and may be used in the invention wherein a single strand of DNA is modified. Moreover, in the presence of Mg²⁺ ions, Cas9 cuts ssDNA bound to the crRNA using its HNH active site independently of PAM.

Optionally, in any configuration, aspect, example or embodiment of the invention, step (a) is carried out by cleavage in one single strand of dsDNA or in ssDNA.

Optionally, in any configuration, aspect, example or embodiment of the invention, step (a) is carried out by combining in a cell the nucleic acid strand, a Cas endonuclease, a crRNA and a tracrRNA (e.g., provided by one or more gRNAs) for targeting the endonuclease to carry out the cleavage, and optionally an insert sequence for homologous recombination with the nucleic acid strand. Instead of an insert sequence, one can use an incoming sequence containing homology arms but no insert sequence, to effect deletion as described above. In an example, the Cas endonuclease is encoded by a nucleotide sequence that has been introduced into the cell. In an example, the gRNA is encoded by a DNA sequence that has been introduced into the cell.

In an example, the method is carried out in the presence of Mg²⁺.

Optionally, in any configuration, aspect, example or embodiment of the invention, step (b) is performed by carrying out homologous recombination with an incoming nucleic acid comprising first and second homology arms, wherein the homology arms are substantially homologous respectively to a sequence extending 5′ from the 5′ end and a sequence extending 3′ from the 3′ end, wherein the second homology arm comprises a PAM sequence such that homologous recombination between the second homology arm and the sequence extending 3′ from the 3′ end produces a sequence comprising a PAM motif in the product of the method. The PAM can be any PAM sequence disclosed herein, for example. Thus, the method produces a modified nucleic acid strand comprising a PAM that can be used for a subsequent nucleic acid modification according to any configuration, aspect, example or embodiment of the invention, wherein a Cas endonuclease is used to cut the nucleic acid. This is useful, for example, for performing sequential endonuclease-mediated homology directed recombination (sEHDR) according to the invention, more particularly sCHDR described below.

Sequential Endonuclease-Mediated Homology Directed Recombination (sEHDR)

The invention further provides:—

A method of sequential endonuclease-mediated homology directed recombination (SEHDR) comprising carrying out the method of any preceding configuration, aspect, example or embodiment of the invention a first time and a second time, wherein endonuclease-mediated cleavage is used in each step (a); wherein the product of the first time is used for endonuclease-mediated cleavage the second time, whereby either (i) first and second nucleotide sequences are deleted the first time and the second times respectively; (ii) a first nucleotide sequence is deleted the first time and a second nucleotide sequence is inserted the second time; (iii) a first nucleotide sequence is inserted the first time and a second nucleotide sequence is deleted the second time; or (iv) first and second nucleotide sequences are inserted the first and second times respectively; optionally wherein the nucleic acid strand modification the second time is within 20, 10, 5, 4, 3, 2 or 1 or less nucleotides of the nucleic acid strand modification the first time or directly adjacent to the nucleic acid strand modification the first time.

For example, the first and second nucleotide sequences are inserted so that they are contiguous after the insertion the second time. Alternatively, the first and second deletions are such that a contiguous sequence has been deleted after the first and second deletions have been performed.

In an embodiment of sEHDR, the invention uses a Cas endonuclease. Thus, there is provided:

A method of sequential Cas-mediated homology directed recombination (sCHDR) comprising carrying out the method of any preceding claim a first time and a second time, wherein Cas endonuclease-mediated cleavage is used in each step (a); wherein step (b) of the first time is carried out performing homologous recombination with an incoming nucleic acid comprising first and second homology arms, wherein the homology arms are substantially homologous respectively to a sequence extending 5′ from the 5′ end and a sequence extending 3′ from the 3′ end, wherein the second homology arm comprises a PAM sequence such that homologous recombination between the second homology arm and the sequence extending 3′ from the 3′ end produces a sequence comprising a PAM motif in the product of the method; wherein the PAM motif of the product of the first time is used for Cas endonuclease-mediated cleavage the second time, whereby either (i) first and second nucleotide sequences are deleted the first time and the second times respectively; (ii) a first nucleotide sequence is deleted the first time and a second nucleotide sequence is inserted the second time; (iii) a first nucleotide sequence is inserted the first time and a second nucleotide sequence is deleted the second time; or (iv) first and second nucleotide sequences are inserted the first and second times respectively; optionally wherein the nucleic acid strand modification the second time is within 20, 10, 5, 4, 3, 2 or 1 or less nucleotides of the nucleic acid strand modification the first time or directly adjacent to the nucleic acid strand modification the first time.

For example, the first and second nucleotide sequences are inserted so that they are contiguous after the insertion the second time. Alternatively, the first and second deletions are such that a contiguous sequence has been deleted after the first and second deletions have been performed.

In an embodiment (First Embodiment), the first time is carried out according to the third configuration of the invention, wherein the incoming nucleic acid comprises no sequence between the first and second homology arms, wherein sequence between the 5′ and 3′ ends is deleted by homologous recombination; and/or the second time is carried out according to the third configuration of the invention, wherein step (b) is performed by carrying out homologous recombination between an incoming nucleic acid comprising first and second homology arms, wherein the homology arms are substantially homologous respectively to a sequence extending 5′ from the 5′ end and a sequence extending 3′ from the 3′ end, wherein the incoming nucleic acid comprises no sequence between the first and second homology arms such that sequence between the 5′ and 3′ ends is deleted by homologous recombination; optionally wherein the second arm comprises a PAM motif such that the product of the second time comprises a PAM motif for use in a subsequent Cas endonuclease-mediated method according to any configuration, aspect, example or embodiment of the invention.

In an embodiment (Second Embodiment), the first time is carried out according to the first or second configuration of the invention, wherein the incoming nucleic acid comprises the insert sequence between the first and second homology arms, wherein the insert sequence is inserted between the 5′ and 3′ ends by homologous recombination; and/or the second time is carried out according to the first or second configuration of the invention, wherein step (b) is performed by carrying out homologous recombination between an incoming nucleic acid comprising first and second homology arms, wherein the homology arms are substantially homologous respectively to a sequence extending 5′ from the 5′ end and a sequence extending 3′ from the 3′ end, wherein the insert sequence is inserted between the 5′ and 3′ ends by homologous recombination; optionally wherein the second arm comprises a PAM motif such that the product of the second time comprises a PAM motif for use in a subsequent Cas endonuclease-mediated method according to any configuration, aspect, example or embodiment of the invention.

In an example, one of said first and second times is carried out as specified in the First Embodiment and the other time is carried out as specified in the Second Embodiment, wherein at least one sequence deletion and at least one sequence insertion is performed.

Optionally, in any configuration, aspect, example or embodiment of the invention, step (a) is carried out by Cas endonuclease-mediated cleavage using a Cas endonuclease, one or more crRNAs and a tracrRNA. For example, the method is carried out in a cell and the crRNA and tracrRNA is introduced into the cell as RNA molecules. For example, the method is carried out in a zygote (e.g., a non-human zygote, e.g., a rodent, rat or mouse zygote) and the crRNA and tracrRNA is injected into zygote. In another embodiment, the crRNA and tracrRNA are encoded by DNA within a cell or organism and are transcribed inside the cell (e.g., an ES cell, e.g., a non-human ES cell, e.g., a rodent, rat or mouse ES cell) or organism to produce the crRNA and tracrRNA. The organism is, for example, a non-human animal or plant or bacterium or yeast or insect. In an embodiment, the tracrRNA is in this way encoded by DNA but one or more crRNAs are introduced as RNA nucleic acid into the cell or organism to effect the method of the invention.

Additionally or alternatively to these examples, the endonuclease may be introduced as a protein or a vector encoding the endonuclease may be introduced into the cell or organism to effect the method of the invention. In another example, the endonuclease is encoded by DNA that is genomically integrated into the cell or organism and is transcribed and translated inside the cell or organism.

In an example, the method of the invention is carried out in an ES cell (e.g., a non-human ES cell, e.g., a rodent, rat or mouse ES cell) that has been pre-engineered to comprise an expressible genomically-integrated Cas endonuclease sequence (or a vector carrying this has been include in the cell). It would be possible to introduce (or encode) a tracrRNA. By introducing a crRNA with a guiding oligo sequence to target the desired area of the cell genome, one can then carry out modifications in the cell genome as per the invention. In an example, a gRNA as described herein is introduced into the ES cell. The genomically-integrated expressible Cas endonuclease sequence can, for example, be constitutively expressed or inducibly expressible. Alternatively or additionally, the sequence may be expressible in a tissue-specific manner in a progeny organism (e.g., a rodent) developed using the ES cell.

The initial ES cell comprising a genomically-integrated expressible Cas endonuclease sequence can be used, via standard techniques, to produce a progeny non-human animal that contains the expressible Cas endonuclease sequence. Thus, the invention provides:

A non-human animal (e.g., a vertebrate, mammal, fish or bird), animal cell, insect, insect cell, plant or plant cell comprising a genomically-integrated expressible Cas endonuclease nucleotide sequence and optionally a tracrRNA and/or a nucleotide sequence encoding a tracrRNA. The Cas endonuclease is, for example, Cas9 or Cys4. In an example, the animal, insect or plant genome comprises a chromosomal DNA sequence flanked by site-specific recombination sites and/or transposon elements (e.g., piggyBac transposon repeat elements), wherein the sequence encodes the endonuclease and optionally one or more gRNAs. As described in the Examples below, recombinase-mediated cassette exchange (RMCE) can be used to insert such a sequence. The transposon elements can be used to excise the sequence from the genome once the endonuclease has been used to perform recombination. The RMCE and/or transposon-mediated excision can be performed in a cell (e.g., an ES cell) that later is used to derive a progeny animal or plant comprising the desired genomic modification.

The invention also provides an ES cell derived or derivable from such an animal, wherein the ES cell comprises a genomically-integrated expressible Cas endonuclease nucleotide sequence. In an example, the ES cell is a rodent, e.g., a mouse or rat ES cell, or is a rabbit, dog, pig, cat, cow, non-human primate, fish, amphibian or bird ES cell.

The invention also provides a method of isolating an ES cell, the method comprising deriving an ES cell from an animal (e.g., a non-human animal, e.g., a rodent, e.g., a rat or a mouse), wherein the animal comprises a genomically-integrated expressible Cas endonuclease nucleotide sequence, as described herein.

In any of these aspects, instead of an ES cell, the cell may be an iPS cell or a totipotent or pluripotent cell. Thus, an iPS or stem cell can be derived from (e.g., a somatic cell of) a human, engineered in vitro to comprise a genomically-integrated expressible Cas endonuclease nucleotide sequence and optionally one or more DNA sequences encoding a tracrRNA or gRNA. The invention, thus, also relates to such a method and to a human iPS or stem cell comprising a genomically-integrated expressible Cas endonuclease nucleotide sequence and optionally one or more DNA sequences encoding a tracrRNA or gRNA. This cell can be used in a method of the invention to carry out genome modification (e.g., to correct a genetic defect, e.g., by replacement of defective sequence with a desired sequence, optionally with subsequent transposon-mediated excision of the endonuclease-encoding sequence). After optional excision of the Cas endonuclease sequence, the iPS cell or stem cell can be introduced into the donor human (or a different human, e.g., a genetic relative thereof) to carry out genetic therapy or prophylaxis. In the alternative, a totipotent or pluripotent human cell is used and then subsequently developed into human tissue or an organ or part thereof. This is useful for providing material for human therapy or prophylaxis or for producing assay materials (eg, for implantation into model non-human animals) or for use in in vitro testing (e.g., of drugs).

In an example the method uses a single guided RNA (gRNA) comprising a crRNA and a tracrRNA. The crRNA comprises an oligonucleotide sequence (“X” in the structure 5′-X-Y-3′ mentioned below) that is chosen to target a desired part of the nucleic acid or genome to be modified. The skilled person will be able readily to select appropriate oligo sequence. In an example, the sequence is from 3 to 100 nucleotides long, eg, from 3 to 50, 40, 30, 25, 20, 15 or 10 nucleotides long, eg, from or 5, 10, 15 or 20 to 100 nucleotides long, eg, from 5, 10, 15 or 20 to 50 nucleotides long.

For example, the gRNA is a single nucleic acid comprising both the crRNA and the tracrRNA. An example of a gRNA comprises the sequence 5′-[oligo]-[UUUUAGAGCUA] (SEQ ID NO: 1)-[LINKER]-[UAGCAAGUUAAAA] (SEQ ID NO: 2)-3′, wherein the LINKER comprises a plurality (e.g., 4 or more, e.g., 4, 5 or 6) nucleotides (e.g., 5′-GAAA-3′).

For example, the crRNA has the structure 5′-X-Y-3′, wherein X is an RNA nucleotide sequence (optionally at least 5 nucleotides long) and Y is a crRNA sequence comprising a nucleotide motif that hybridises with a motif comprised by the tracrRNA, wherein X is capable of hybridising with a nucleotide sequence 5′ of the desired site of the 5′ cut end, e.g., extending 5′ from the desired site of the 5′ cut.

In an example, Y is 5′-N1UUUUAN2N3GCUA-3′ (SEQ ID NO: 3), wherein each of N1-3 is a A, U, C or G and/or the tracrRNA comprises the sequence (in 5′ to 3′ orientation) UAGCM1UUAAAAM2 (SEQ ID NO: 4, when M1 is, e.g., 5 nucleotides), wherein M1 is spacer nucleotide sequence and M2 is a nucleotide; e.g., N1-G, N2=G and N3=A. The spacer sequence is, e.g., 5, 4, 3, 2 or 1 RNA nucleotides in length (e.g., AAG in 5′ to 3′ orientation). M2 is, for example, a A,U, C or G (e.g., M2 is a G). In an embodiment, a chimaeric gRNA is used which comprises a sequence 5′-X-Y-Z-3′, wherein X and Y are as defined above and Z is a tracrRNA comprising the sequence (in 5′ to 3′ orientation) UAGCM1UUAAAAM2 (SEQ ID NO: 4), wherein M1 is spacer nucleotide sequence and M2 is a nucleotide. In an example, Z comprises the sequence 5′-UAGCAAGUUAAAA-3′ (SEQ ID NO: 2), e.g., Z is 5′-UAGCAAGUUAAAAUAAGGCUAGUCCG-3′ (SEQ ID NO: 5). In an example, the gRNA has the sequence:

(SEQ ID NO: 5)
5′-GUUUUAGAGCUAGAAAUAGCAAGUUAAAAUAAGGCUAGUCCGUUAUC
AACUUGAAAAAGUGGCACCGAGUCGGUGC-3′.

When it is desired to use the present invention to insert an exogenous sequence into the nucleic acid to be modified, the exogenous sequence can be provided on linear or circular nucleic acid (e.g., DNA). Typically, the exogenous sequence is flanked by homology arms that can undergo homologous recombination with sequences 5′ and 3′ respectively of the site where the exogenous sequence is to be inserted. The skilled person is familiar with choosing homology arms for homologous recombination.

The invention can be used in a method of producing a transgenic organism, e.g., any organism recited herein. For example, the organism can be a non-human organism used as an assay model to test a pharmaceutical drug or to express an exogenous protein or a part thereof (e.g., a human protein target knocked-in into a non-human animal assay organism). In another example, the invention has been used to knock-out an endogenous sequence (e.g., a target protein) in an organism, such as a non-human organism. This can be useful to assess the effect (phenotype) of the knock-out and thus to assess potential drug targets or proteins implicated in disease. In one example, the organism is a non-human animal (e.g., a vertebrate, mammal, bird, fish, rodent, mouse, rat or rabbit) in which a human target protein has been knocked-in using the invention. Optionally, the invention has been used to knock out an orthologous or homologous endogenous target of the organism (eg, an endogenous target sequence has been replaced at the endogenous position by an orthologous or homologous human target sequence). In this way, an assay model can be produced for testing pharmaceutical drugs that act via the human target.

In an embodiment, the organism is a non-human vertebrate that expresses human antibody variable regions whose genome comprises a replacement of an endogenous target with an orthologous or homologous human sequence. In an example, the method of the invention is used to produce an Antibody-Generating Vertebrate or Assay Vertebrate as disclosed in WO2013061078, the disclosure of which, and specifically including the disclosure of such Vertebrates, their composition, manufacture and use, is included specifically herein by reference as though herein reproduced in its entirety and for providing basis for claims herein.

In an example, an exogenous regulatory element is knocked-in using the method. For example, it is knocked-in to replace an endogenous regulatory element.

In one aspect, the invention provides a method of producing a cell or a transgenic non-human organism (e.g., any non-human organism recited herein), the method comprising
(a) carrying out the method of any in any configuration, aspect, example or embodiment of the invention to (i) knock out a target nucleotide sequence in the genome of a first cell and/or (ii) knock in an insert nucleotide sequence into the genome of a first cell, optionally wherein the insert sequence replaces a target sequence in whole or in part at the endogenous location of the target sequence in the genome; wherein the cell or a progeny thereof can develop into a non-human organism or cell; and
(b) developing the cell or progeny into a non-human organism or a non-human cell.

In an example, the organism or cell is homozygous for the modification (i) and/or (ii).

In an example, the cell is an ES cell, iPS cell, totipotent cell or pluripotent cell. In an example, the cell is a non-human vertebrate cell or a human cell in vitro. In an example, the cell is a plant, yeast, insect or bacterial cell.

In an example, the cell or organism is a rodent (e.g., a mouse or rat) cell or a rabbit, bird, fish, chicken, non-human primate, monkey, pig, dog, Camelid, shark, sheep, cow or cat cell.

In an example, the target sequence is an endogenous sequence comprising all or part of a regulatory element or encoding all or part of a protein.

In an example, the insert sequence is a synthetic sequence; or comprises a sequence encoding all or part of a protein from a species other than the species from which the first cell is derived; or comprises a regulatory element from said first species. This is useful to combine genes with new regulatory elements.

In an example, the insert sequence encodes all or part of a human protein or a human protein subunit or domain. For example, the insert sequence encodes a cell membrane protein, secreted protein, intracellular protein, cytokine, receptor protein (e.g., Fc receptor protein, such as FcRn or a FcY receptor protein), protein of the human immune system or domain thereof (e.g., an Ig protein or domain, such as an antibody or TCR protein or domain, or a MHC protein), a hormone or growth factor.

The invention also provides:

A cell (e.g., an isolated or purified cell, eg, a cell in vitro, or any cell disclosed herein) or a non-human organism (e.g., any organism disclosed herein) whose genome comprises a modification comprising a non-endogenous nucleotide sequence flanked by endogenous nucleotide sequences, wherein the cell or organism is obtainable by the method of any configuration, aspect, example or embodiment of the invention, and wherein the non-endogenous sequence is flanked 3′ and/or 5′ by (e.g., within 20, 10, 5, 4, 3, 2 or 1 or less nucleotides of, or directly adjacent to) a Cas PAM motif; wherein the cell is not comprised by a human; and one, more or all of (a) to (d) applies (a) the genome is homozygous for the modification; or comprises the modification at one allele and is unmodified by Cas-mediated homologous recombination at the other allele;
(b) the non-endogenous sequence comprises all or part of a regulatory element or encodes all or part of a protein;
(c) the non-endogenous sequence is at least 20, 30, 40, 50, 60, 70, 80, 90, 100, 150, 200, 300, 400, 500, 600, 700, 800 or 900 nucleotides, or at least 1, 2, 3, 5, 10, 20, 50 or 100 kb long;
(d) the non-endogenous sequence replaces an orthologous or homologous sequence in the genome.

The cell can be a human cell, or included in human tissue but not part of a human being. For example, the cell is a human cell in vitro.

In an example, the non-endogenous sequence is a human sequence.

In an example, the PAM motif is any PAM disclosed herein or comprises a sequence selected from CCN, TCN, TTC, AWG, CC, NNAGNN, NGGNG GG, NGG, WGG, CWT, CTT and GAA. For example, the motif is a Cas9 PAM motif. For example, the PAM is NGG. In another example, the PAM is GG.

In an example, there is a PAM motif no more than 10 nucleotides (e.g., 3 nucleotides) 3′ and/or 5′ of the non-endogenous sequence.

In an example, the PAM motif is recognised by a Streptococcus Cas9.

In an example, the cell or organism is a non-human vertebrate cell or a non-human vertebrate that expresses one or more human antibody heavy chain variable domains (and optionally no heavy chain variable domains of a non-human vertebrate species). For example, the organism is an Antibody-Generating Vertebrate or Assay Vertebrate disclosed in WO2013061078.

In an example, the cell or organism is a non-human vertebrate cell or a non-human vertebrate that expresses one or more human antibody kappa light chain variable domains (and optionally no kappa light chain variable domains of a non-human vertebrate species).

In an example, the cell or organism is a non-human vertebrate cell or a non-human vertebrate that expresses one or more human antibody lambda light chain variable domains (and optionally no kappa light chain variable domains of a non-human vertebrate species).

In an example, the non-endogenous sequence encodes a human Fc receptor protein or subunit or domain thereof (e.g., a human FcRn or FcY receptor protein, subunit or domain).

In an example, the non-endogenous sequence comprises one or more human antibody gene segments, an antibody variable region or an antibody constant region.

In an example, the insert sequence is a human sequence that replaces or supplements an orthologous non-human sequence.

The invention also provides:

A monoclonal or polyclonal antibody prepared by immunisation of a vertebrate (e.g., mouse or rat) of the invention (or produced by a method of the invention) with an antigen.

The invention also provides:

A method of isolating an antibody that binds a predetermined antigen, the method comprising
(a) providing a vertebrate (optionally a mouse or rat) of the invention (or produced by a method of the invention);
(b) immunising said vertebrate with said antigen;
(c) removing B lymphocytes from the vertebrate and selecting one or more B lymphocytes expressing antibodies that bind to the antigen;
(d) optionally immortalising said selected B lymphocytes or progeny thereof, optionally by producing hybridomas therefrom; and
(e) isolating an antibody (eg, and IgG-type antibody) expressed by the B lymphocytes.

In an example, the method comprises the step of isolating from said B lymphocytes nucleic acid encoding said antibody that binds said antigen; optionally exchanging the heavy chain constant region nucleotide sequence of the antibody with a nucleotide sequence encoding a human or humanised heavy chain constant region and optionally affinity maturing the variable region of said antibody; and optionally inserting said nucleic acid into an expression vector and optionally a host.

In an example, the method comprises making a mutant or derivative of the antibody produced by the method.

The invention provides the use of an isolated, monoclonal or polyclonal antibody described herein, or a mutant or derivative antibody thereof that binds said antigen, in the manufacture of a composition for use as a medicament.

The invention provides the use of an isolated, monoclonal or polyclonal antibody described herein, or a mutant or derivative antibody thereof that binds said antigen for use in medicine.

The invention provides a nucleotide sequence encoding an antibody described herein, optionally wherein the nucleotide sequence is part of a vector.

The invention provides a pharmaceutical composition comprising the antibody or antibodies described herein and a diluent, excipient or carrier.

The invention provides an ES cell, a non-human animal or a non-human blastocyst comprising an expressible genomically-integrated nucleotide sequence encoding a Cas endonuclease (e.g., a Cas9 or Cys4) and optionally an expressible genomically-integrated nucleotide sequence encoding a tracrRNA or a gRNA. For example, the ES cell is any ES cell type described herein.

In an example of the cell, animal or blastocyst, the endonuclease sequence is constitutively expressible.

In an example of the cell, animal or blastocyst, the endonuclease sequence is inducibly expressible.

In an example of the cell, animal or blastocyst, the endonuclease sequence is expressible in a tissue-specific manner in the animal or a progeny thereof, or in a non-human animal that is a progeny of the cell or blastocyst.

In an example, the cell, animal or blastocyst comprises one or more gRNAs or an expressible nucleotide sequence encoding a gRNA or a plurality of expressible nucleotide sequences each encoding a different gRNA.

The invention provides the use of the cell, animal or blastocyst in a method according to any configuration, aspect, embodiment or example of the invention.

An aspect provides an antibody produced by the method of the invention, optionally for use in medicine, eg, for treating and/or preventing a medical condition or disease in a patient, e.g., a human.

An aspect provides a nucleotide sequence encoding the antibody of the invention, optionally wherein the nucleotide sequence is part of a vector. Suitable vectors will be readily apparent to the skilled person, eg, a conventional antibody expression vector comprising the nucleotide sequence together in operable linkage with one or more expression control elements.

An aspect provides a pharmaceutical composition comprising the antibody of the invention and a diluent, excipient or carrier, optionally wherein the composition is contained in an IV container (e.g., and IV bag) or a container connected to an IV syringe.

An aspect provides the use of the antibody of the invention in the manufacture of a medicament for the treatment and/or prophylaxis of a disease or condition in a patient, e.g., a human.

In a further aspect the invention relates to humanised antibodies and antibody chains produced according to the present invention, both in chimaeric and fully humanised form, and use of said antibodies in medicine. The invention also relates to a pharmaceutical composition comprising such an antibody and a pharmaceutically acceptable carrier or other excipient.

Antibody chains containing human sequences, such as chimaeric human-non human antibody chains, are considered humanised herein by virtue of the presence of the human protein coding regions region. Fully human antibodies may be produced starting from DNA encoding a chimaeric antibody chain of the invention using standard techniques.

Methods for the generation of both monoclonal and polyclonal antibodies are well known in the art, and the present invention relates to both polyclonal and monoclonal antibodies of chimaeric or fully humanised antibodies produced in response to antigen challenge in non human-vertebrates of the present invention.

In a yet further aspect, chimaeric antibodies or antibody chains generated in the present invention may be manipulated, suitably at the DNA level, to generate molecules with antibody-like properties or structure, such as a human variable region from a heavy or light chain absent a constant region, for example a domain antibody; or a human variable region with any constant region from either heavy or light chain from the same or different species; or a human variable region with a non-naturally occurring constant region; or human variable region together with any other fusion partner. The invention relates to all such chimaeric antibody derivatives derived from chimaeric antibodies identified according to the present invention.

In a further aspect, the invention relates to use of animals of the present invention in the analysis of the likely effects of drugs and vaccines in the context of a quasi-human antibody repertoire.

The invention also relates to a method for identification or validation of a drug or vaccine, the method comprising delivering the vaccine or drug to a mammal of the invention and monitoring one or more of: the immune response, the safety profile; the effect on disease.

The invention also relates to a kit comprising an antibody or antibody derivative as disclosed herein and either instructions for use of such antibody or a suitable laboratory reagent, such as a buffer, antibody detection reagent.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Cong L, Ran F A, Cox D, Lin S, Barretto R, Habib N, Hsu P D, Wu X, Jiang W, Marraffini L A et al: Multiplex genome engineering using CRISPR/Cas systems. Science 2013, 339(6121):819-823.
2. Wang H, Yang H, Shivalila C S, Dawlaty M M, Cheng A W, Zhang F, Jaenisch R: One-step generation of mice carrying mutations in multiple genes by CRISPR/Cas-mediated genome engineering. Cell 2013, 153(4):910-918.
3. Mali P, Yang L, Esvelt K M, Aach J, Guell M, DiCarlo J E, Norville J E, Church G M: RNA-guided human genome engineering via Cas9. Science 2013, 339(6121):823-826.
4. Gaj T, Gersbach C A, Barbas C F, 3rd: ZFN, TALEN, and CRISPR/Cas-based methods for genome engineering. Trends Biotechnol 2013, 31(7):397-405.
5. Perez-Pinera P, Ousterout D G, Gersbach C A: Advances in targeted genome editing. Curr Opin Chem Biol 2012, 16(3-4):268-277.
6. Shah S A, Erdmann S, Mojica F J, Garrett R A: Protospacer recognition motifs: Mixed identities and functional diversity. RNA Biol 2013, 10(5).
7. Haurwitz R E, Sternberg S H, Doudna J A: Csy4 relies on an unusual catalytic dyad to position and cleave CRISPR RNA. EMBO J 2012, 31(12): 2824-2832.
8. Yusa K, Zhou L, Li M A, Bradley A, Craig N L: A hyperactive piggyBac transposase for mammalian applications. Proc Natl Acad Sci USA 2011, 108(4):1531-1536.
9. Qiao J, Oumard A, Wegloehner W, Bode J: Novel tag-and-exchange (RMCE) strategies generate master cell clones with predictable and stable transgene expression properties. J Mol Biol 2009, 390(4):579-594.
10. Oumard A, Qiao J, Jostock T, Li J, Bode J: Recommended Method for Chromosome Exploitation: RMCE-based Cassette-exchange Systems in Animal Cell Biotechnology. Cytotechnology 2006, 50(1-3):93-108.

The present invention is described in more detail in the following non limiting exemplification.

### EXAMPLES

### Example 1: Precise DNA Modifications

### (a) Use of Nickase for HDR

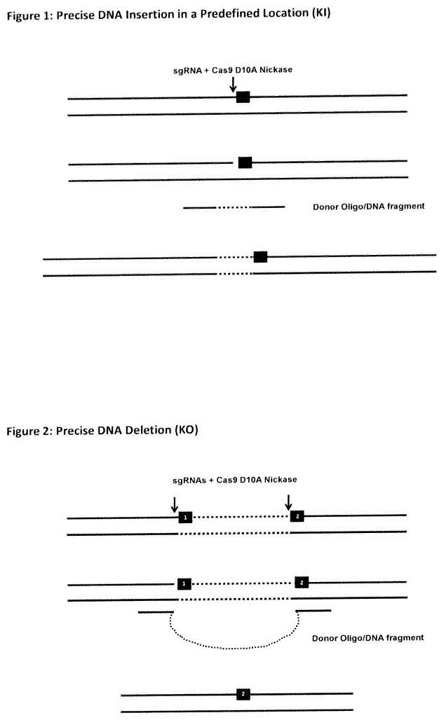

It has been reported that the Cas9 nuclease can be converted into a nickase through the substitution of an aspartate to alanine (D10A) in the RuvCI domain of SpCas9 (Cong et al). It is noteworthy that DNA single-stranded breaks are preferentially repaired through the HDR pathway. The Cas9 D10A nickase, when in a complex with mature crRNA:tracrRNA, can specifically induce DNA nicking at a precise location. With this in mind, we propose extending the application of the CRISPR/Cas system by creating a nick in a given location in a genome using Cas9 D10A nickase and then exploiting the HDR pathway for inserting a single-stranded DNA fragment (endogenous or exogenous) which will contain DNA homology flanking the nick. Typically for recombineering 50 bp is enough for efficient recombination) flanking the nicked DNA junction to bring in and insert a given DNA in a precision location; similar size homology will be used with the present example (FIG. 1). Guide RNA (gRNA) will be design individually per target protospacer sequence or incorporated into a single CRISPR array encoding for 2 or more spacer sequences allowing multiplex genome editing from a single CRSPR array.

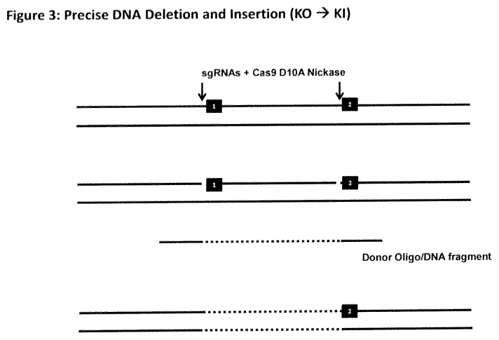

In a separate setting, two gRNA or a single CRISPR array encoding multiple spacer sequence can be designed flanking a gene or a region of interest and with the association of Cas9 D10A nickase, two separate single-stranded breaks can be induced. This in association with a single-stranded DNA fragment containing DNA homology to the 5′ breakpoint junction of the first DNA nick and DNA homology to the 3′ breakpoint junction of the second nick the region in between the two single stranded DNA nick can be precisely deleted (FIG. 2). In an another setting, two separate gRNA or a multiplex single CRISPR array can be designed flanking a gene or a region of interest and with the association of Cas9 D10A nickase two separate single-stranded breaks can be induced. In this case the intruding single stranded DNA fragment can contain DNA sequence from either endogenous or exogenous source containing sequence for a known gene, regulatory element promoter etc. This single-stranded DNA fragment (or double stranded DNA) can be brought together to replace the DNA region of interest flanked by DNA nick by arming it with DNA homology from the 5′ region of the first nick and 3′ region from the second nick (FIG. 3). Due to the high efficiency of the CRISPR/Cas system to cleave DNA, the above proposed strategy will not require introduction of any selection marker thus creating exact seamless genome editing in a precise and defined manner. As an option, a selection marker can be included flanked by PiggyBac LTRs to allow for the direct selection of correctly modified clones. Once the correct clones have been identified, the selection marker can be removed conveniently through the expression of hyperactive piggyBac transposase (Yusa K, Zhou L, Li M A, Bradley A, Craig N L: A hyperactive piggyBac transposase for mammalian applications. Proc Natl Acad Sci USA 2011, 108(4): 1531-1536). Furthermore, the above approaches can be applied to ES cells, mammalian cells, yeast cells, bacterial cells, plant cells as well as directly performing in zygotes to expedite the process of homozygeous genome engineering in record time. It would be also possible to multiplex this system to generate multiple simultaneous DNA insertions (KI), deletions (KO) and the sequential deletion and insertion (KO→KI).

### Example 2: Recycling PAM for Sequential Insertions or Deletions

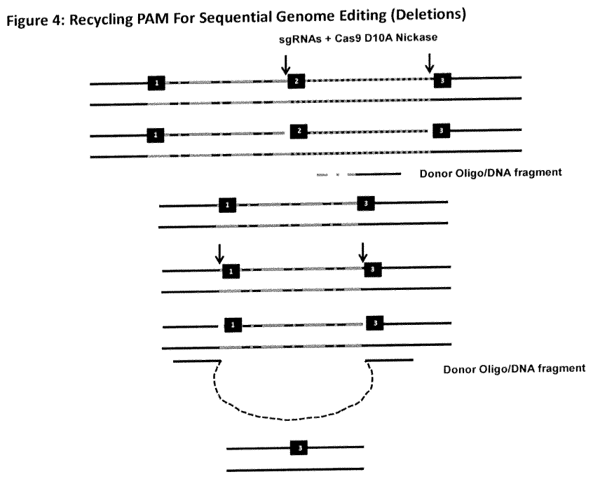

In certain settings it may be useful to edit a genome by chromosome walking. Using any of the three examples outlined above, it could be possible to carry out sequential genome editing in a stepwise fashion whereby the PAM sequence used in a previous round of CRISPR/Cas mediated genome editing, can be re-used to carry out multiple rounds of genome editing such as deletions, insertions or the simultaneous deletion and insertion. An example of sequential deletion whereby the PAM sequence from the previous genome editing step is recycled is shown in FIG. 4. Using the PAM recycling approach, it is possible to carry out sequential insertions as well as sequential simultaneous deletion and insertion.

### Example 3: Rapid Insertion of Lox Sites Using CRISPR/Cas System

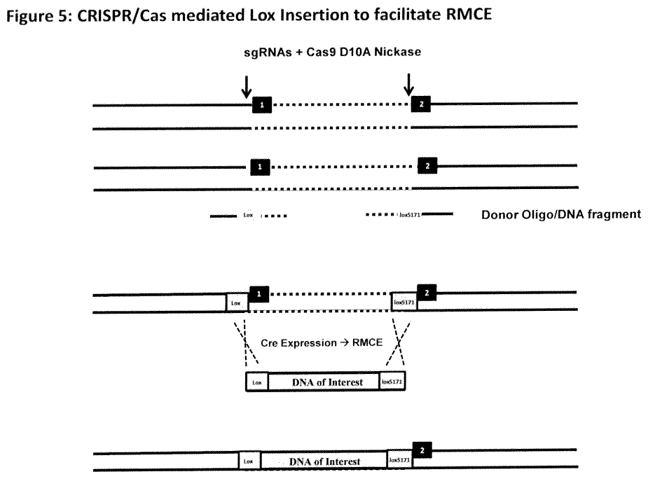

Targeting Efficiency Using Conventional Homologous Recombination Methods in ES cells is low. In a different setting, the CRISPR/Cas system can be used to rapidly and efficiently introduce lox sites or other recombinase recognition sequence such as Frt in a defined location to act as a landing pad for genome editing using recombinase mediated cassette exchange (RMCE) (Qiao J, Oumard A, Wegloehner W, Bode J: Novel tag-and-exchange (RMCE) strategies generate master cell clones with predictable and stable transgene expression properties. J Mol Biol 2009, 390(4): 579-594; and Oumard A, Qiao J, Jostock T, Li J, Bode J: Recommended Method for Chromosome Exploitation: RMCE-based Cassette-exchange Systems in Animal Cell Biotechnology. Cytotechnology 2006, 50(1-3):93-108). Once the lox sites are introduced into the genome, inversion, deletion or cassette exchange to delete and introduce DNA fragment varying in size at this site can be efficiently conducted via expression of Cre recombinase. An example of CRISPR/Cas mediated lox insertion followed by RMCE is shown in FIG. 5. The RMCE step can be used to invert the region flanked by lox site or to delete this region as well as to simultaneously delete and insert DNA of interest in this region. Furthermore, the RMCE step can be adapted for carrying out multiple sequential rounds of RMCE (sRMCE).

### Example 4

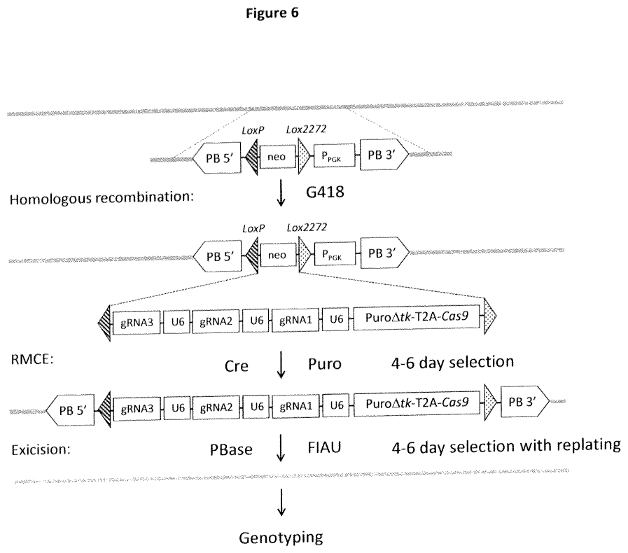

Reference is made to FIG. 6. A piggyBac transposon harbouring a PGK promoter-driven loxP/mutant lox-flanked neoR gene is targeted into an ES cell genome by standard homologous recombination. The targeted clones can be selected by G418. This provides a landing pad for the following recombinase-mediated cassette exchange (RMCE). Such an ES clone can be used a parental cells for any modification further. A cassette containing the loxP/mutant lox-flanked promoterless PuroATK-T2A-Cas9 and U6 polymerase III promoter-driven guide RNA (gRNA) genes are inserted into the landing pad through transient cre expression. The gRNA genes can be one or more than one which target to the same gene or different genes. The inserted clones can be selected with puromycin and confirmed by junction PCRs. During the selection, the expression of Cas9 and gRNAs from the inserted cassette results in more efficient gene targeting or modification than transient expression of the Cas9 and gRNA can achieve. Following 4-6 day selection, the whole modified cassette is excised by the transient expression of piggyBac transposase (PBase). The final ES cell clones would not contain any Cas9 or gRNA sequence. The clones with homozygous modified genes would be confirmed by PCR and sequence.

The main feature of this invention is to control the Cas9 and gRNA expression in certain time to be sufficient to generate efficient targeting rates.

### Example 5: Methodology

### Reconstructing CRISPR/Cas Vector System (Nuclease)

The CRISPR/Cas genome editing system has been reconstructed in vitro and exemplified in mouse embryonic stem cells using vector pX330 containing humanised S. pyogenes (hSpCsn1) (Cong et al). The CRISPR/Cas system can be reconstructed as described in Cong et al using synthetic DNA strings and DNA assembly. In the present example, the entire DNA assembly would constitute a 6006 bp fragment containing 45 bp homology to pBlueScript KS+ vector 5′ to the EcoRV cutting site, Human U6 promoter, two Bbsl restriction sites for cloning in the spacer sequence which fuses to a chimeric guided RNA sequence, chicken beta-actin promoter with 3 FLAG, nuclear localisation signal (NLS) followed by hSpCsn1 sequence and another NLS, bGH polyA, inverted terminal repeat sequence and finally another 45 bp homology to pBlueScript KS+3′ to the EcoRV cutting site. This 6006 bp stretch of DNA will be synthetized as 7 individual DNA fragments where each fragment will have a 45 bp overlap to the adjacent DNA fragment to allow DNA assembly. The DNA sequence of these fragments is shown below in the order of assembly.

### Fragment 1A (1340 bp)

(SEQ ID NO: 7)
GGTACCGGGCCCCCCCTCGAGGTCGACGGTATCGATAAGCTTGATGAGGG
CCTATTTCCCATGATTCCTTCATATTTGCATATACGATACAAGGCTGTTA
GAGAGATAATTGGAATTAATTTGACTGTAAACACAAAGATATTAGTACAA
AATACGTGACGTAGAAAGTAATAATTTCTTGGGTAGTTTGCAGTTTTAAA
ATTATGTTTTAAAATGGACTATCATATGCTTACCGTAACTTGAAAGTATT
TCGATTTCTTGGCTTTATATATCTTGTGGAAAGGACGAAACACCGGGTCT
TCGAGAAGACCTGTTTTAGAGCTAGAAATAGCAAGTTAAAATAAGGCTAG
TCCGTTATCAACTTGAAAAAGTGGCACCGAGTCGGTGCTTTTTTGTTTTA
GAGCTAGAAATAGCAAGTTAAAATAAGGCTAGTCCGTTTTTAGCGCGTGC
GCCAATTCTGCAGACAAATGGCTCTAGAGGTACCCGTTACATAACTTACG
GTAAATGGCCCGCCTGGCTGACCGCCCAACGACCCCCGCCCATTGACGTC
AATAGTAACGCCAATAGGGACTTTCCATTGACGTCAATGGGTGGAGTATT
TACGGTAAACTGCCCACTTGGCAGTACATCAAGTGTATCATATGCCAAGT
ACGCCCCCTATTGACGTCAATGACGGTAAATGGCCCGCCTGGCATTGTGC
CCAGTACATGACCTTATGGGACTTTCCTACTTGGCAGTACATCTACGTAT
TAGTCATCGCTATTACCATGGTCGAGGTGAGCCCCACGTTCTGCTTCACT
CTCCCCATCTCCCCCCCCTCCCCACCCCCAATTTTGTATTTATTTATTTT
TTAATTATTTTGTGCAGCGATGGGGGCGGGGGGGGGGGGGGGGCGCGCGC
CAGGCGGGGCGGGGCGGGGCGAGGGGCGGGGCGGGGCGAGGCGGAGAGGT
GCGGCGGCAGCCAATCAGAGCGGCGCGCTCCGAAAGTTTCCTTTTATGGC
GAGGCGGCGGCGGCGGCGGCCCTATAAAAAGCGAAGCGCGCGGCGGGCGG
GAGTCGCTGCGACGCTGCCTTCGCCCCGTGCCCCGCTCCGCCGCCGCCTC
GCGCCGCCCGCCCCGGCTCTGACTGACCGCGTTACTCCCACAGGTGAGCG
GGCGGGACGGCCCTTCTCCTCCGGGCTGTAATTAGCTGAGCAAGAGGTAA
GGGTTTAAGGGATGGTTGGTTGGTGGGGTATTAATGTTTAATTACCTGGA
GCACCTGCCTGAAATCACTTTTTTTCAGGTTGGACCGGTGCCACCATGGA
CTATAAGGACCACGACGGAGACTACAAGGATCATGATATT.

### Fragment 2 (852 bp)

(SEQ ID NO: 8)
ATGGACTATAAGGACCACGACGGAGACTACAAGGATCATGATATTGATTA
CAAAGACGATGACGATAAGATGGCCCCAAAGAAGAAGCGGAAGGTCGGTA
TCCACGGAGTCCCAGCAGCCGACAAGAAGTACAGCATCGGCCTGGACATC
GGCACCAACTCTGTGGGCTGGGCCGTGATCACCGACGAGTACAAGGTGCC
CAGCAAGAAATTCAAGGTGCTGGGCAACACCGACCGGCACAGCATCAAGA
AGAACCTGATCGGAGCCCTGCTGTTCGACAGCGGCGAAACAGCCGAGGCC
ACCCGGCTGAAGAGAACCGCCAGAAGAAGATACACCAGACGGAAGAACCG
GATCTGCTATCTGCAAGAGATCTTCAGCAACGAGATGGCCAAGGTGGACG
ACAGCTTCTTCCACAGACTGGAAGAGTCCTTCCTGGTGGAAGAGGATAAG
AAGCACGAGCGGCACCCCATCTTCGGCAACATCGTGGACGAGGTGGCCTA
CCACGAGAAGTACCCCACCATCTACCACCTGAGAAAGAAACTGGTGGACA
GCACCGACAAGGCCGACCTGCGGCTGATCTATCTGGCCCTGGCCCACATG
ATCAAGTTCCGGGGCCACTTCCTGATCGAGGGCGACCTGAACCCCGACAA
CAGCGACGTGGACAAGCTGTTCATCCAGCTGGTGCAGACCTACAACCAGC
TGTTCGAGGAAAACCCCATCAACGCCAGCGGCGTGGACGCCAAGGCCATC
CTGTCTGCCAGACTGAGCAAGAGCAGACGGCTGGAAAATCTGATCGCCCA
GCTGCCCGGCGAGAAGAAGAATGGCCTGTTCGGAAACCTGATTGCCCTGA
GC.

### Fragment 3 (920 bp)

(SEQ ID NO: 9)
GGCGAGAAGAAGAATGGCCTGTTCGGAAACCTGATTGCCCTGAGCCTGGG
CCTGACCCCCAACTTCAAGAGCAACTTCGACCTGGCCGAGGATGCCAAAC
TGCAGCTGAGCAAGGACACCTACGACGACGACCTGGACAACCTGCTGGCC
CAGATCGGCGACCAGTACGCCGACCTGTTTCTGGCCGCCAAGAACCTGTC
CGACGCCATCCTGCTGAGCGACATCCTGAGAGTGAACACCGAGATCACCA
AGGCCCCCCTGAGCGCCTCTATGATCAAGAGATACGACGAGCACCACCAG
GACCTGACCCTGCTGAAAGCTCTCGTGCGGCAGCAGCTGCCTGAGAAGTA
CAAAGAGATTTTCTTCGACCAGAGCAAGAACGGCTACGCCGGCTACATTG
ACGGCGGAGCCAGCCAGGAAGAGTTCTACAAGTTCATCAAGCCCATCCTG
GAAAAGATGGACGGCACCGAGGAACTGCTCGTGAAGCTGAACAGAGAGGA
CCTGCTGCGGAAGCAGCGGACCTTCGACAACGGCAGCATCCCCCACCAGA
TCCACCTGGGAGAGCTGCACGCCATTCTGCGGCGGCAGGAAGATTTTTAC
CCATTCCTGAAGGACAACCGGGAAAAGATCGAGAAGATCCTGACCTTCCG
CATCCCCTACTACGTGGGCCCTCTGGCCAGGGGAAACAGCAGATTCGCCT
GGATGACCAGAAAGAGCGAGGAAACCATCACCCCCTGGAACTTCGAGGAA
GTGGTGGACAAGGGCGCTTCCGCCCAGAGCTTCATCGAGCGGATGACCAA
CTTCGATAAGAACCTGCCCAACGAGAAGGTGCTGCCCAAGCACAGCCTGC
TGTACGAGTACTTCACCGTGTATAACGAGCTGACCAAAGTGAAATACGTG
ACCGAGGGAATGAGAAAGCC.

### Fragment 4 (920 bp)

(SEQ ID NO: 10)
CGAGCTGACCAAAGTGAAATACGTGACCGAGGGAATGAGAAAGCCCGCCT
TCCTGAGCGGCGAGCAGAAAAAGGCCATCGTGGACCTGCTGTTCAAGACC
AACCGGAAAGTGACCGTGAAGCAGCTGAAAGAGGACTACTTCAAGAAAAT
CGAGTGCTTCGACTCCGTGGAAATCTCCGGCGTGGAAGATCGGTTCAACG
CCTCCCTGGGCACATACCACGATCTGCTGAAAATTATCAAGGACAAGGAC
TTCCTGGACAATGAGGAAAACGAGGACATTCTGGAAGATATCGTGCTGAC
CCTGACACTGTTTGAGGACAGAGAGATGATCGAGGAACGGCTGAAAACCT
ATGCCCACCTGTTCGACGACAAAGTGATGAAGCAGCTGAAGCGGCGGAGA
TACACCGGCTGGGGCAGGCTGAGCCGGAAGCTGATCAACGGCATCCGGGA
CAAGCAGTCCGGCAAGACAATCCTGGATTTCCTGAAGTCCGACGGCTTCG
CCAACAGAAACTTCATGCAGCTGATCCACGACGACAGCCTGACCTTTAAA
GAGGACATCCAGAAAGCCCAGGTGTCCGGCCAGGGCGATAGCCTGCACGA
GCACATTGCCAATCTGGCCGGCAGCCCCGCCATTAAGAAGGGCATCCTGC
AGACAGTGAAGGTGGTGGACGAGCTCGTGAAAGTGATGGGCCGGCACAAG
CCCGAGAACATCGTGATCGAAATGGCCAGAGAGAACCAGACCACCCAGAA
GGGACAGAAGAACAGCCGCGAGAGAATGAAGCGGATCGAAGAGGGCATCA
AAGAGCTGGGCAGCCAGATCCTGAAAGAACACCCCGTGGAAAACACCCAG
CTGCAGAACGAGAAGCTGTACCTGTACTACCTGCAGAATGGGCGGGATAT
GTACGTGGACCAGGAACTGG.

### Fragment 5 (920 bp)

(SEQ ID NO: 11)
ACTACCTGCAGAATGGGCGGGATATGTACGTGGACCAGGAACTGGACATC
AACCGGCTGTCCGACTACGATGTGGACCATATCGTGCCTCAGAGCTTTCT
GAAGGACGACTCCATCGACAACAAGGTGCTGACCAGAAGCGACAAGAACC
GGGGCAAGAGCGACAACGTGCCCTCCGAAGAGGTCGTGAAGAAGATGAAG
AACTACTGGCGGCAGCTGCTGAACGCCAAGCTGATTACCCAGAGAAAGTT
CGACAATCTGACCAAGGCCGAGAGAGGCGGCCTGAGCGAACTGGATAAGG
CCGGCTTCATCAAGAGACAGCTGGTGGAAACCCGGCAGATCACAAAGCAC
GTGGCACAGATCCTGGACTCCCGGATGAACACTAAGTACGACGAGAATGA
CAAGCTGATCCGGGAAGTGAAAGTGATCACCCTGAAGTCCAAGCTGGTGT
CCGATTTCCGGAAGGATTTCCAGTTTTACAAAGTGCGCGAGATCAACAAC
TACCACCACGCCCACGACGCCTACCTGAACGCCGTCGTGGGAACCGCCCT
GATCAAAAAGTACCCTAAGCTGGAAAGCGAGTTCGTGTACGGCGACTACA
AGGTGTACGACGTGCGGAAGATGATCGCCAAGAGCGAGCAGGAAATCGGC
AAGGCTACCGCCAAGTACTTCTTCTACAGCAACATCATGAACTTTTTCAA
GACCGAGATTACCCTGGCCAACGGCGAGATCCGGAAGCGGCCTCTGATCG
AGACAAACGGCGAAACCGGGGAGATCGTGTGGGATAAGGGCCGGGATTTT
GCCACCGTGCGGAAAGTGCTGAGCATGCCCCAAGTGAATATCGTGAAAAA
GACCGAGGTGCAGACAGGCGGCTTCAGCAAAGAGTCTATCCTGCCCAAGA
GGAACAGCGATAAGCTGATC.

### Fragment 6 (789 bp)

(SEQ ID NO: 12)
AGCAAAGAGTCTATCCTGCCCAAGAGGAACAGCGATAAGCTGATCGCCAG
AAAGAAGGACTGGGACCCTAAGAAGTACGGCGGCTTCGACAGCCCCACCG
TGGCCTATTCTGTGCTGGTGGTGGCCAAAGTGGAAAAGGGCAAGTCCAAG
AAACTGAAGAGTGTGAAAGAGCTGCTGGGGATCACCATCATGGAAAGAAG
CAGCTTCGAGAAGAATCCCATCGACTTTCTGGAAGCCAAGGGCTACAAAG
AAGTGAAAAAGGACCTGATCATCAAGCTGCCTAAGTACTCCCTGTTCGAG
CTGGAAAACGGCCGGAAGAGAATGCTGGCCTCTGCCGGCGAACTGCAGAA
GGGAAACGAACTGGCCCTGCCCTCCAAATATGTGAACTTCCTGTACCTGG
CCAGCCACTATGAGAAGCTGAAGGGCTCCCCCGAGGATAATGAGCAGAAA
CAGCTGTTTGTGGAACAGCACAAGCACTACCTGGACGAGATCATCGAGCA
GATCAGCGAGTTCTCCAAGAGAGTGATCCTGGCCGACGCTAATCTGGACA
AAGTGCTGTCCGCCTACAACAAGCACCGGGATAAGCCCATCAGAGAGCAG
GCCGAGAATATCATCCACCTGTTTACCCTGACCAATCTGGGAGCCCCTGC
CGCCTTCAAGTACTTTGACACCACCATCGACCGGAAGAGGTACACCAGCA
CCAAAGAGGTGCTGGACGCCACCCTGATCCACCAGAGCATCACCGGCCTG
TACGAGACACGGATCGACCTGTCTCAGCTGGGAGGCGAC.

### Fragment 7 (535 bp)

(SEQ ID NO: 13)
GGCCTGTACGAGACACGGATCGACCTGTCTCAGCTGGGAGGCGACAAAAG
GCCGGCGGCCACGAAAAAGGCCGGCCAGGCAAAAAAGAAAAAGTAAGAAT
TCCTAGAGCTCGCTGATCAGCCTCGACTGTGCCTTCTAGTTGCCAGCCAT
CTGTTGTTTGCCCCTCCCCCGTGCCTTCCTTGACCCTGGAAGGTGCCACT
CCCACTGTCCTTTCCTAATAAAATGAGGAAATTGCATCGCATTGTCTGAG
TAGGTGTCATTCTATTCTGGGGGGTGGGGTGGGGCAGGACAGCAAGGGGG
AGGATTGGGAAGAGAATAGCAGGCATGCTGGGGAGCGGCCGCAGGAACCC
CTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCT
CAGTGAGCGAGCGAGCGCGCAGCTGCCTGCAGGGGCGCCTATCGAATTCC
TGCAGCCCGGGGGATCCACTAGTTCTAGAGCGGCC.

To reconstruct the CRISPR/Cas system described in Cong et al the above DNA fragments in addition to EcoRV linearised pBlueScript KS+ vector will be assembled using Gibson Assembly kit (NEB Cat No. E5510S). As an alternative approach, the 6006 bp fragment can be assembled by assembly PCR by mixing molar ratio of the individual DNA fragments together and using the DNA mixture as PCR template. The assembled PCR product can then be cloned directly into pBlueScript vector or a standard cloning vector system such as a TOPO TA cloning kit (Invitrogen).

### Reconstructing CRISPR/Cas Vector System (D10A Nickase)

The D10A nickase version of the CRISPR/Cas system can be conveniently reconstructed by assembling the above fragments where fragment 2 is replaced with fragment 2A which contains the D10A substitution (See sequence below).

### Fragment 2A (852 bp)

(SEQ ID NO: 14)
ATGGACTATAAGGACCACGACGGAGACTACAAGGATCATGATATTGATTA
CAAAGACGATGACGATAAGATGGCCCCAAAGAAGAAGCGGAAGGTCGGTA
TCCACGGAGTCCCAGCAGCCGACAAGAAGTACAGCATCGGCCTGgccATC
GGCACCAACTCTGTGGGCTGGGCCGTGATCACCGACGAGTACAAGGTGCC
CAGCAAGAAATTCAAGGTGCTGGGCAACACCGACCGGCACAGCATCAAGA
AGAACCTGATCGGAGCCCTGCTGTTCGACAGCGGCGAAACAGCCGAGGCC
ACCCGGCTGAAGAGAACCGCCAGAAGAAGATACACCAGACGGAAGAACCG
GATCTGCTATCTGCAAGAGATCTTCAGCAACGAGATGGCCAAGGTGGACG
ACAGCTTCTTCCACAGACTGGAAGAGTCCTTCCTGGTGGAAGAGGATAAG
AAGCACGAGCGGCACCCCATCTTCGGCAACATCGTGGACGAGGTGGCCTA
CCACGAGAAGTACCCCACCATCTACCACCTGAGAAAGAAACTGGTGGACA
GCACCGACAAGGCCGACCTGCGGCTGATCTATCTGGCCCTGGCCCACATG
ATCAAGTTCCGGGGCCACTTCCTGATCGAGGGCGACCTGAACCCCGACAA
CAGCGACGTGGACAAGCTGTTCATCCAGCTGGTGCAGACCTACAACCAGC
TGTTCGAGGAAAACCCCATCAACGCCAGCGGCGTGGACGCCAAGGCCATC
CTGTCTGCCAGACTGAGCAAGAGCAGACGGCTGGAAAATCTGATCGCCCA
GCTGCCCGGCGAGAAGAAGAATGGCCTGTTCGGAAACCTGATTGCCCTGA
GC.

The substituted aspartate to alanine is highlighted in bold and underlined.

### Target (Spacer) Sequence Cloning

The target spacer sequence can be cloned into the above CRISPR/Cas vector system via the Bbsl restriction sites located upstream of the chimeric guided RNA sequence. The spacer sequence can be ordered as oligo pairs and annealed together with overhangs as shown below to allow direct cloning into Bbsl linearised CRISPR/Cas vector using standard molecular biology protocols.

Sequence of an example oligo pair with spacer sequence:

(SEQ ID NO: 15)
5′-CACCGNNNNNNNNNNNNNNNNNNN-3′.
(SEQ ID NO: 16)
3′-CNNNNNNNNNNNNNNNNNNNCAAA-5′.

The 4 bp overhang sequence underlined is required to be included in the spacer oligos to facilitate cloning into the Bbsl restriction site in the CRISPR/Cas vector. Using this approach, any spacer sequence can be conveniently cloned into the CRISPR/Cas vector.

### Reconstructing CRISPR/Cas System for One-Step Generation of Transgenic Animals

In order to reconstitute a CRISPR/Cas system for one-step generation of transgenic animal as described in Wang et al (Wang H, Yang H, Shivalila C S, Dawlaty M M, Cheng A W, Zhang F, Jaenisch R: One-step generation of mice carrying mutations in multiple genes by CRISPR/Cas-mediated genome engineering. Cel/2013, 153(4):910-918) where direct embryo injection is used, the above detailed CRISPR/Cas vector system needs to be modified to incorporate a T7 polymerase promoter to the Cas9 coding sequence. In addition, the gRNA needs to be removed and synthetised separately by annealing oligos or produced synthetically (See below for an example T7-spacer sequence fused to chimeric guided RNA sequence—T7-gRNA). Note, ideally the spacer sequence will be designed in a unique region of a given chromosome to minimise off-target effect and also the respective protospacer genomic sequence needs to have a PAM at the 3′-end.

### Example T7-gRNA Sequence

(SEQ ID NO: 17)
TTAATACGACTCACTATAGGNNNNNNNNNNNNNNNNNNNNGTTTTAGAGC
TAGAAATAGCAAGTTAAAATAAGGCTAGTCCGTTATCAACTTGAAAAAGT
GGCACCGAGTCGGTGCTTTTTT.

The underlined 20 bp of N's depicts the spacer sequence for a given target DNA.

To reconstruct the one-step CRISPR/Cas system, the above detailed DNA fragments (Fragments 2, 3, 4, 5, 6 & 7) can be assembled together where fragment 1A (containing 45 bp homology to pBlueScript KS+ vector 5′ to the EcoRV restriction site, human U6 promoter, Bbsl restriction sites, chimeric guided RNA sequence and chicken b-actin promoter) is replaced with fragment 1, which only contains 45 bp homology to pBlueScript KS+ vector and the DNA sequence for T7 polymerase promoter with 45 bp homology to fragment 2. This will create the nuclease version of the CRISPR/Cas system for one-step generation of transgenic animals. To create the nickase version, fragment 2 can be replaced with fragment 2A as detailed above and then fragments 1, 2A, 3, 4, 5, 6 and 7 can be assembled together either by Gibson assembly or by assembly PCR.

### Fragment 1 (111 bp)

(SEQ ID NO: 18)
GGTACCGGGCCCCCCCTCGAGGTCGACGGTATCGATAAGCTTGATAATAC
GACTCACTATAGGGAGAATGGACTATAAGGACCACGACGGAGACTACAAG
GATCATGATATT.

### Preparation of Oligo/DNA Fragments for HDR-Mediated Repair

DNA oligos ranging from 15 bp and upwards in excess of >125 bp will be synthetised through Sigma Custom Oligo synthesis Service. The oligos can contain any sequence such as a defined mutation, introduced restriction sites or a sequence of interest including recombination recognition sequence such as loxP or derivatives thereof, Frt and derivatives thereof or PiggyBac LTR or any other transposon elements or regulatory elements including enhancers, promoter sequence, reporter gene, selection markets and tags. The oligo design will incorporate DNA homology to the region where Cas9 mediates double-stranded DNA break or DNA nick. The size of the homology will range from a few base pairs (2-5 bp) to upwards and in excess of 80 bp. Larger DNA fragments (>100 bp ranging up to several kilobases) will be prepared either synthetically (GeneArt) or by PCR. The DNA fragment will be synthetised either with or without flanked NLS or only with a single NLS and either with or without a promoter (e.g., 17 polymerase promoter). The DNA can be prepared as a single stranded DNA fragment using either the capture biotinylated target DNA sequence method (Invitrogen: DYNABEADS M-270 Streptavidin) or any other standard and established single stranded DNA preparation methodology. The single stranded DNA can be prepared for microinjection by IVT as described above and the mRNA co-injected with Cas9 mRNA and gRNA. The DNA fragment can also be co-injected as a double stranded DNA fragment. The DNA fragment will be flanked by DNA homology to the site where Cas9 mediates double-stranded DNA break or DNA nick. The DNA homology can range from a few base pairs (2-5 bp) and up to or in excess of several kilobases. The DNA fragment can be used to introduce any endogenous or exogenous DNA.

HDR-mediated repair can also be done in ES cells following CRISPR/Cas-mediated DNA cleavage. The above mentioned donor oligo or DNA fragment can be co-transfected into ES cells along with the CRISPR/Cas expression vector.

Production of Cas9 mRNA and gRNA

Vector containing the T7 polymerase promoter with the coding region of humanised Cas9 will be PCR amplified using oligos Cas9-F and Cas9-R. The T7-Cas9 PCR product can be gel extracted and the DNA purified using Qiagen gel extraction kit. The purified T7-Cas9 DNA will be used for in vitro transcription (IVT) using mMESSAGE mMACHINE T7 Ultra Kit (Life Technologies Cat No. AM1345). The vector containing the T7-gRNA can be PCR amplified using oligos gRNA-F and gRNA-R and once again the PCR products gel purified. IVT of the T7-gRNA will be carried out using MEGAshortscript T7 Kit (Life Technologies Cat No. AM1354) and the gRNA purified using MEGAclear Kit (Life Technologies Cat No. AM1908) and eluted in RNase-free water.

Cas9-F:
(SEQ ID NO: 19)
TTAATACGACTCACTATAGG
Cas9-R:
(SEQ ID NO: 20)
GCGAGCTCTAGGAATTCTTAC
gRNA-F:
(SEQ ID NO: 21)
TTAATACGACTCACTATAGG
gRNA-R:
(SEQ ID NO: 22)
AAAAAAGCACCGACTCGGTGCCAC

### ES Cell Transfection Procedure

Mouse embryonic stem cells AB2.1 and derivatives of this line will be used for transfecting the mammalian codon optimised Cas9 and sgRNA from a single expression vector or from separate vectors if desired. AB2.1 ES cells will be cultured on a PSNL76/7/4 MEF feeder layer in M-15: Knockout DMEM (Gibco, no pyruvate, high glucose, 15% FBS, 1xGPS, 1xBME) with standard ES cell culturing techniques. Transfection of CRISPR/Cas expression vector along with the optional addition of a donor oligo or DNA fragment will be done by electroporation using the Amaxa 4D-Nucleofector® Protocol (Lonza). A plasmid expressing PGK-Puro will also be optionally co-transfected to promote transfection efficiency. After transfection ES cells will be plated back onto feeder plates and Puromycin (2 μg/ml) will be added 72 hours post transfection for 7 days after which colonies will be picked and genotyped by PCR. Positive colonies will be further cultured and expanded on feeder layer and selection markers where necessary will be excised using a PiggyBac transposon system. This will be done by electroporation of ES cells with a plasmid containing HyPbase using the Amaxa 4D-Nucleofector® Protocol (Lonza). The ES cell will be plated back onto feeder plates. ES cells will be passaged 2-3 days post transfection and after a further 2-3 days the ES cells will be plated out at different cells densities (1:10, 1:20, 1:100 and 1:300) and FIAU (2 μg/ml) selection will be added 24 hours after replating. Colonies will be picked and analysed by PCR genotyping after 7-10 days on selection media. Positive clones will be further cultured and expanded on feeder layer and sent for zygote (blastocyst) microinjection.

### Microinjection of Mouse Zygotes

### Materials and Reagents

M2 (Sigma M7167)
Embryo Max KSOM (Speciality media MR-020P-F)
Hyaluronidase (Sigma H4272)
Mineral Oil (Sigma, M-8410)

### Possible Donor Strains:

S3F/S3F;KF3/KF3
S3F/S3F;K4/K4
S7F/S7F
K5F/K5F

### Preparation of Zygotes and Microinjection:

The protocol is as described in: A.Nagy Et al. Manipulating the Mouse Embryo 3^{rd} Edition. Chapter 7, Protocols 7-1, 7-6, 7-10, 7-11.Cold Spring Harbor Laboratory Press.

### In Brief:

1. Zygotes are harvested from E0.5dpc (day post-coitum) superovulated female mice.
2. The zygotes are incubated in hyaluronidase to disperse cumulus cells.
3. Zygotes are collected and transferred to several drops of M2 medium to rinse off the hyaluronidase solution and debris. Zygotes are placed into KSOM Media and incubated at 37° C., 5% CO2 until required.
4. Zygote quality is assessed and zygotes with normal morphology are selected for injection, these are placed in KSOM media and incubated at 37ºC, 5% CO2 until required.

### Microinjection Set Up:

Injection procedures are performed on a Nikon Eclipse Ti inverted microscope with Eppendorf micromanipulators and an Eppendorf femtojet injection system. A slide is prepared by adding a large drop ˜200 microlitres of M2 into the centre.

### Microinjection:

Place an appropriate number of zygotes onto the slide. Examine the zygotes and select only those with normal morphology (2 distinct pronuclei are visible). Whilst holding a zygote with a male pronucleus closest to the injection pipette, carefully push the injection pipette through the zona pellucida into the pronucleus, apply injection pressure, the pronucleus should visibly swell, remove the injection pipette quickly. The injected zygote can be placed down while the rest are injected.

At the end of the injection session all viable injected zygotes should be placed into prepared dishes containing drops of KSOM and incubated until ready to surgically implant. They are incubated for 2-3 hours before surgically implanting into pseudo pregnant females. Pups will be born 21 days later.

### Example 6

### Single Copy Cas9 Expression in ES Cells

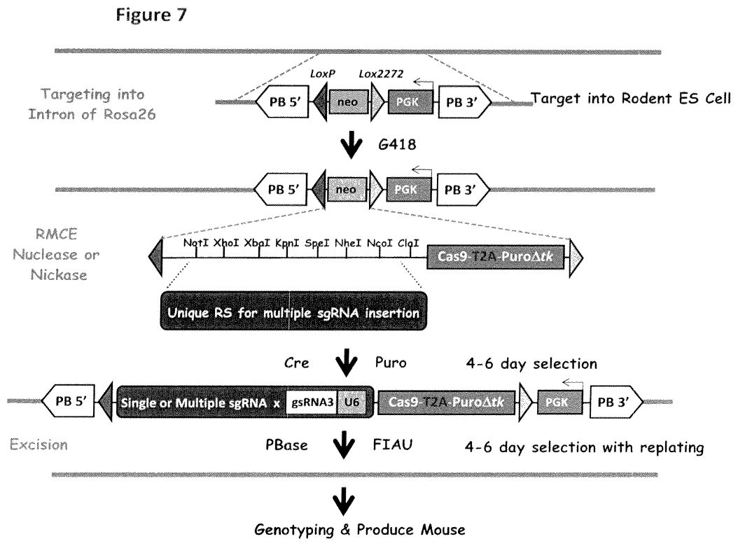

Reference is made to FIG. 7.

1. A landing pad consisting of a PiggyBac transposon element with the following features will be targeted into mouse ES cells (e.g., 129-derived ES cells, such as AB2.1 ES cells; Baylor College of Medicine, Texas, USA) and selected for on G418. The PiggyBac transposon element will contain neomycin resistance gene flanked by loxP and lox2272. It will also have a geneless PGK promoter. In this example, the landing pad will be targeted into the introgenic region of Rosa26 gene located on chromosome 6, but it could be targeted elsewhere. Targeting this landing pad in the Rosa26 gene will provide a universal ES cell line for precisely inserting any desired DNA fragment including DNA fragments containing Cas9, mutant Cas9 or any other gene of interest via RMCE with high efficiency. Targeting Rosa26 is beneficial since the targeted construct will be inserted as a single copy (unlike random integration elsewhere) and is unlikely to produce an unwanted phenotypic effect.

Note. This landing pad can be inserted into any gene in any chromosome or indeed in any eukaryotic or mammalian cell line, e.g., a human, insect, plant, yeast, mouse, rat, rabbit, rodent, pig, dog, cat, fish, chicken or bird cell line, followed by generation of the respective transgenic organism expressing Cas9.

### Rosa 26 Locus

Ubiquitous expression of transgene in mouse embryonic stem cell can be achieved by gene targeting to the ROSA26 locus (also known as: gene trap ROSA 26 or Gt(ROSA)26) by homologous recombination (Ref. (a) and (b) below). The genomic coordinates for mouse C57BL/6J Rosa26 gene based on Ensemble release 73—September 2013 is: Chromosome 6: 113,067,428-113,077,333; reverse strand.

The Rosa26 locus can also be used to as a recipient location to knock-in a transgene. In our example we have use the Rosa26 locus to knock-in the landing pad vector by targeting through homologous recombination into the intronic region located between exons 2 and 3 of mouse strain 129-derived embryonic stem cells using approx. 3.1 kb homology arms. The homology arms were retrieved by recombineering from a BAC Clone generated from mouse strain 129. The sequence of the Rosa26 homology arms used for targeting is given below.

### Sequence of Rosa26 5′ Homology Arm

(SEQ ID NO: 23)
CACATTTGGTCCTGCTTGAACATTGCCATGGCTCTTAAAGTCTTAATTAA
GAATATTAATTGTGTAATTATTGTTTTTCCTCCTTTAGATCATTCCTTGA
GGACAGGACAGTGCTTGTTTAAGGCTATATTTCTGCTGTCTGAGCAGCAA
CAGGTCTTCGAGATCAACATGATGTTCATAATCCCAAGATGTTGCCATTT
ATGTTCTCAGAAGCAAGCAGAGGCATGATGGTCAGTGACAGTAATGTCAC
TGTGTTAAATGTTGCTATGCAGTTTGGATTTTTCTAATGTAGTGTAGGTA
GAACATATGTGTTCTGTATGAATTAAACTCTTAAGTTACACCTTGTATAA
TCCATGCAATGTGTTATGCAATTACCATTTTAAGTATTGTAGCTTTCTTT
GTATGTGAGGATAAAGGTGTTTGTCATAAAATGTTTTGAACATTTCCCCA
AAGTTCCAAATTATAAAACCACAACGTTAGAACTTATTTATGAACAATGG
TTGTAGTTTCATGCTTTTAAAATGCTTAATTATTCAATTAACACCGTTTG
TGTTATAATATATATAAAACTGACATGTAGAAGTGTTTGTCCAGAACATT
TCTTAAATGTATACTGTCTTTAGAGAGTTTAATATAGCATGTCTTTTGCA
ACATACTAACTTTTGTGTTGGTGCGAGCAATATTGTGTAGTCATTTTGAA
AGGAGTCATTTCAATGAGTGTCAGATTGTTTTGAATGTTATTGAACATTT
TAAATGCAGACTTGTTCGTGTTTTAGAAAGCAAAACTGTCAGAAGCTTTG
AACTAGAAATTAAAAAGCTGAAGTATTTCAGAAGGGAAATAAGCTACTTG
CTGTATTAGTTGAAGGAAAGTGTAATAGCTTAGAAAATTTAAAACCATAT
AGTTGTCATTGCTGAATATCTGGCAGATGAAAAGAAATACTCAGTGGTTC
TTTTGAGCAATATAACAGCTTGTTATATTAAAAATTTTCCCCACAGATAT
AAACTCTAATCTATAACTCATAAATGTTACAAATGGATGAAGCTTACAAA
TGTGGCTTGACTTGTCACTGTGCTTGTTTTAGTTATGTGAAAGTTTGGCA
ATAAACCTATGTCCTAAATAGTCAAACTGTGGAATGACTTTTTAATCTAT
TGGTTTGTCTAGAACAGTTATGTTGCCATTTGCCCTAATGGTGAAAGAAA
AAGTGGGGAGTGCCTTGGCACTGTTCATTTGTGGTGTGAACCAAAGAGGG
GGGCATGCACTTACACTTCAAACATCCTTTTGAAAGACTGACAAGTTTGG
GTCTTCACAGTTGGAATTGGGCATCCCTTTTGTCAGGGAGGGAGGGAGGG
AGGGAGGCTGGCTTGTTATGCTGACAAGTGTGATTAAATTCAAACTTTGA
GGTAAGTTGGAGGAACTTGTACATTGTTAGGAGTGTGACAATTTGGACTC
TTAATGATTTGGTCATACAAAATGAACCTAGACCAACTTCTGGAAGATGT
ATATAATAACTCCATGTTACATTGATTTCACCTGACTAATACTTATCCCT
TATCAATTAAATACAGAAGATGCCAGCCATCTGGGCCTTTTAACCCAGAA
ATTTAGTTTCAAACTCCTAGGTTAGTGTTCTCACTGAGCTACATCCTGAT
CTAGTCCTGAAAATAGGACCACCATCACCCCCAAAAAAATCTCAAATAAG
ATTTATGCTAGTGTTTCAAAATTTTAGGAATAGGTAAGATTAGAAAGTTT
TAAATTTTGAGAAATGGCTTCTCTAGAAAGATGTACATAGTGAACACTGA
ATGGCTCCTAAAGAGCCTAGAAAACTGGTACTGAGCACACAGGACTGAGA
GGTCTTTCTTGAAAAGCATGTATTGCTTTACGTGGGTCACAGAAGGCAGG
CAGGAAGAACTTGGGCTGAAACTGGTGTCTTAAGTGGCTAACATCTTCAC
AACTGATGAGCAAGAACTTTATCCTGATGCAAAAACCATCCAAACAAACT
AAGTGAAAGGTGGCAATGGATCCCAGGCTGCTCTAGAGGAGGACTTGACT
TCTCATCCCATCACCCACACCAGATAGCTCATAGACTGCCAATTAACACC
AGCTTCTAGCCTCCACAGGCACCTGCACTGGTACACATAATTTCACACAA
ACACAGTAAGAAGCCTTCCACCTGGCATGGTATTGCTTATCTTTAGTTCC
CAACACTTGGGAGGCAGAGGCCAGCCAGGGCTATGTGACAAAAACCTTGT
CTAGAGGAGAAACTTCATAGCTTATTTCCTATTCACGTAACCAGGTTAGC
AAAATTTACCAGCCAGAGATGAAGCTAACAGTGTCCACTATATTTGTAGT
GTTTTAAGTCAATTTTTTAAATATACTTAATAGAATTAAAGCTATGGTGA
ACCAAGTACAAACCTGGTGTATTAACTTGAGAACTTAGCATAAAAAGTAG
TTCATTTGTTCAGTAAATATTAAATGCTTACTGGCAAAGATTATGTCAGG
AACTTGGTAAATGGTGATGAAACAATCATAGTTGTACATCTTGGTTCTGT
GATCACCTTGGTTTGAGGTAAAAGTGGTTCCTTTGATCAAGGATGGAATT
TTAAGTTTATATTCAATCAATAATGTATTATTTTGTGATTGCAAAATTGC
CTATCTAGGGTATAAAACCTTTAAAAATTTCATAATACCAGTTCATTCTC
CAGTTACTAATTCCAAAAAGCCACTGACTATGGTGCCAATGTGGATTCTG
TTCTCAAAGGAAGGATTGTCTGTGCCCTTTATTCTAATAGAAACATCACA
CTGAAAATCTAAGCTGAAAGAAGCCAGACTTTCCTAAATAAATAACTTTC
CATAAAGCTCAAACAAGGATTACTTTTAGGAGGCACTGTTAAGGAACTGA
TAAGTAATGAGGTTACTTATATAATGATAGTCCCACAAGACTATCTGAGG
AAAAATCAGTACAACTCGAAAACAGAACAACCAGCTAGGCAGGAATAACA
GGGCTCCCAAGTCAGGAGGTCTATCCAACACCCTTTTCTGTTGAGGGCCC
CAGACCTACATATTGTATACAAACAGGGAGGTGGGTGATTTTAACTCTCC
TGAGGTAC

### Sequence of Rosa26 3′ Homology Arm

(SEQ ID NO: 24)
CTTGGTAAATCTTTGTCCTGAGTAAGCAGTACAGTGTACAGTTTACATTT
TCATTTAAAGATACATTAGCTCCCTCTACCCCCTAAGACTGACAGGCACT
TTGGGGGTGGGGAGGGCTTTGGAAAATAACGCTTCCATACACTAAAAGAG
AAATTTCTTTAATTAGGCTTGTTGGTTCCATACATCTACTGGTGTTTCTA
CTACTTAGTAATATTATAATAGTCACACAAGCATCTTTGCTCTGTTTAGG
TTGTATATTTATTTTAAGGCAGATGATAAAACTGTAGATCTTAAGGGATG
CTTCTGCTTCTGAGATGATACAAAGAATTTAGACCATAAAACAGTAGGTT
GCACAAGCAATAGAATATGGCCTAAAGTGTTCTGACACTTAGAAGCCAAG
CAGTGTAGGCTTCTTAAGAAATACCATTACAATCACCTTGCTAGAAATCA
AGCATTCTGGAGTGGTCAAGCAGTGTAACCTGTACTGTAAGTTACTTTTC
TGCTATTTTTCTCCCAAAGCAAGTTCTTTATGCTGATATTTCCAGTGTTA
GGAACTACAAATATTAATAAGTTGTCTTCACTCTTTTCTTTACCAAGGAG
GGTCTCTTCCTTCATCTTGATCTGAAGGATGAACAAAGGCTTGAGCAGTG
CGCTTTAGAAGATAAACTGCAGCATGAAGGCCCCCGATGTTCACCCAGAC
TACATGGACCTTTCGCCACACATGTCCCATTCCAGATAAGGCCTGGCACA
CACAAAAAACATAAGTCATTAGGCTACCAGTCTGATTCTAAAACAACCTA
AAATCTTCCCACTTAAATGCTATGGGTGGTGGGTTGGAAAGTTGACTCAG
AAAATCACTTGCTGTTTTTAGAGAGGATCTGGGTTCAGTTTCTGATACAT
TGTGGCTTACAACTATAACTCCAGTTCTAGGGGGTCCATCCAACATCCTC
TTCTGTTGAGGGCACCAAATAAATGTATTGTGTACAAACAGGGAGGTGAG
TGATTTAACTCTCGTGTATAGTACCTTGGTAAAACATTTCTTGTCCTGAG
TAAGCAGTACAGCTCTGCCTGTCCCTGGTCTACAGACACGGCTCATTTCC
CGAAGGCAAGCTGGATAGAGATTCCAATTTCTCTTCTTGGATCCCATCCT
ATAAAAGAAGGTCAAGTTTAATCTATTGCAAAAGGTAAATAGGTAGTTTC
TTACATGAGACAAGAACAAATCTTAGGTGTGAAGCAGTCATCTTTTACAG
GCCAGAGCCTCTATTCTATGCCAATGAAGGAAACTGTTAGTCCAGTGTTA
TAGAGTTAGTCCAGTGTATAGTTTTCTATCAGAACACTTTTTTTTTAAAC
AACTGCAACTTAGCTTATTGAAGACAAACCACGAGTAGAAATCTGTCCAA
GAAGCAAGTGCTTCTCAGCCTACAATGTGGAATAGGACCATGTAATGGTA
CAGTGAGTGAAATGAATTATGGCATGTTTTTCTGACTGAGAAGACAGTAC
AATAAAAGGTAAACTCATGGTATTTATTTAAAAAGAATCCAATTTCTACC
TTTTTCCAAATGGCATATCTGTTACAATAATATCCACAGAAGCAGTTCTC
AGTGGGAGGTTGCAGATATCCCACTGAACAGCATCAATGGGCAAACCCCA
GGTTGTTTTTCTGTGGAGACAAAGGTAAGATATTTCAATATATTTTCCCA
AGCTAATGAGATGGCTCAGCAAATAATGGTACTGGCCATTAAGTCTCATG
ACCTGAGCTTGATCCTCAGGGACCATGTGGTACAAGGAGAGACCTAAATC
CTTCAGTTGGACTTCAATCTTCTACCCTCATGTCCACACACAAATAAATA
CAATAAAAAACATTCTGCAGTCTGAATTTCTAAAGGTTGTTTTTCTAAAA
AGAAATGTTAAAGTAACATAGGAAGAAATATGTCCATAACTGAAATACAA
GTTTTTTAAATGGTTAAGACTGGTTTTCAAAGGATGTATGGTTAAGAAAA
TACCAGGGAAAATGAGCTTACATGTAAAAAAGTGTCTAAAAGGCCAGAGA
AATGACCCAGCTGGCAAAGGTGTCTGCCCTAAGCCAGACAAAAGGAATTT
GATTCACAGGAAGAAGAGACCCAACTCTCACTAGTTATCCTCTGACTTCC
ACACCATGACACAGCTCCATGGCACTCTCAGGCCCCCACACATATACAGA
TATAAACAGAAACCTAATCCACCAGCCTTCAGAAGCAAAGCAATTGGAGG
ATTTAAACAGGCCATGGCTACTAATAGAGATAACTGGTAGTTTAAAAGTT

ATGGTAATGACTTTCATGCTTCTTTCAACTCATATTGTTCTAAATAATTA
ATTTGGTTTTTCAAGGCAGGGTTTCTCTGTGTAGTTCTGGCTGTCCTGGA
ACTCACTCTGTAGACCAGGCTGGCCTTGAACTCAGATCCATCTGCCTCTG
GAATAAGGGCACGTGCGTGCCTTTTCTACATAACAAAACCTATACTATAA
CAAAACCTATACCATACTGTACCGTTTTGGGAAAAGACAAAAAATAATGA
ACAAAAAAGGAGAAATAACATTCCAATAAAGTATGGAAATGGTAGTTAAA
TTAATTACAAATGTTTTTCAGTAAATTAGATGTGACTTCTCATACTGTTC
ATTTGGCTATAATGATACCACAAAGCACTGGGGGTGAATAATAATTCCAA
GTCAGTAGGGAGAGAGACTTGAAAAGATGCAATGCAATCATTGAAGTTAA
ACTTACCCATCTTTAATCTGGCTCTTAGTCAATAGAGATGAGATGTTATT
TGCTGCTCTGTTCACTGCCAGTGGGTTATTGTCCCCAGCAATATGGTAAC
AGTGAGACCACTCAGTAGCCCCCTATGAGACAGGAGTGTTGGTTAAACAT
GCCACAAGAGAAAAGGGAAAAGTCACTATGGCCAACTCTCAGTAACATGG
CAATCCGTGCCATTCATTTCCTTGCCAGAAATGTCTTCCCTGTTCTTCTG
CCTACTGAACTTTCACCCACTAGAAATGTGGCTCCAATGTCATCCACTAT
GACATCAATGTCAGCGCTAGAAGCACTTTGCACACCTCTGTTGCTGACTT
AG

### Reference

a) Pablo Perez-Pinera, David G. Ousterout, Matthew T. Brown and Charles A. Gersbach (2012) Gene targeting to the ROSA26 locus directed by engineered zinc finger nucleases. Nucleic Acids Research, 2012, Vol. 40, No. 8 3741-3752
b) Peter Hohenstein, Joan Slight, Derya Deniz Ozdemir, Sally F Burn, Rachel Berry and Nicholas D Hastie (2008) High-efficiency Rosa26 knock-in vector construction for Cre-regulated overexpression and RNAi. PathoGenetics 2008, 1:3

2. A recombinase mediated cassette exchange (RMCE)-enabled vector containing a promoterless puromycin-delta-tk with in-frame fusion of T2A at the C-terminus following by either Cas9 or mutant Cas9 nucleotide sequence and a series of unique restriction sites flanked by loxP and lox2272 will allow for the direct targeting of this vector into the landing pad by Cre-mediated RMCE. As is known, T2A allows ribosomal skipping during translation. The insertion of the coding sequence of T2A between two genes results in two products (one gene, one transcript but two proteins expressed, in this case the Cas9 and selection marker). ES clones containing the correctly inserted DNA fragment can be directly selected on puromycin. This approach also advantageously ensures single copy expression of Cas9 as suppose to a random integration or transient expression approach. Insertion of the RMCE enabled vector into the desired locus containing the landing pad can be selected directly as the PGK promoter in the landing pad will drive the transcription of the promoterless Puro-Delta-Tk and Cas9. Since the Puro-delta-Tk is in the same transcriptional unit as Cas9, ES clones selected on puromycin will ensure expression of Cas9.

3. The above strategy allows for three separate approaches to express the sgRNA designed for disrupting (mutation through indel formation, deletion or deletion followed by insertion) gene of interest.
a. The above ES cell line containing Cas9 can be used for generating transgenic mice with either constitutively expressed Cas9 or modified for inducible Cas9 expression or indeed tissue specific Cas9 expression for example expression of Cas9 at an embryo stage using Nanog-, Pou5f1- or SoxB promoter-specifc Cas9 expression. Such derived mouse line expressing Cas9 can be used for genome editing in a streamline fashion whereby in vitro transcribed sgRNA can be easily injected into embryos obtained from such transgenic mice. This will enhance the efficiency of generating mouse lines with the desired homozygous genotype and thus will dramatically reduce the number of animals required.
b. sgRNA can be transfected directly into the ES cells expressing Cas9 and thus avoids the requirement for cloning into the RMCE enabled vector single or multiple sgRNA. This approach will allow multiple sgRNA to be inserted into the ES cells simultaneously very rapidly.
c. Multiple sgRNA can be cloned directly into the multiple cloning site of the RMCE enabled vector (i.e., using a plurality of different restriction endonuclease sites) to allow single copy expression of the guide-RNA. This approach may be useful for limiting off-target effects particularly relevant for those genes with high sequence homology within the genome.

4. ES cells expressing Cas9 and sgRNA can be selected for directly on medium containing puromycin. Selection on puromycin for 4-6 days will allow for the desired location to be mutated or disrupted and the advantage of manipulating ES cells is that individual clones can be analysed by PCR followed by sequencing for the desired mutation. Only correctly mutated ES cell clones can be processed further whereby inserted DNA element introduced through insertion of the landing pad and the subsequent insertion of the RMCE vector can be completely removed leaving the ES cell devoid of any alteration other than the intended mutation induced by the action of Cas9 and the sgRNA. This can be done through transiently expressing PBase transposon followed by selection on FIAU. Removal of the constitutively expressed Cas9 with only the minimal length of time required to induce mutation in the presence of sgRNA will reduce or eliminate the possibility of Cas9 inducing unwanted mutations.

5. ES Clones containing the desired mutation can be injected into blastocyst to generate transgenic mice.

In Table 1, sequence identification numbers for sequences from top to bottom in the column under the header “CRISPR Consensus sequences” are SEQ ID NO: 25, SEQ ID NO: 26, SEQ ID NO: 27,

SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36 and SEQ ID NO: 37. The sequence identification numbers for sequences from top to bottom in the column under the header “Leaders” are SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60 and SEQ ID NO: 61.

TABLE 1
PAM conservation in repeats and
leaders for various CRISPR types
(reproduced from Short motif sequences
determine the targets of the prokaryotic
CRISPR defence system F.J.M. Mojica, C.
Diez-Villaseñor, J. Garcia-Martinez, C.
Almendros Microbiology(2009), 155, 733-740)

| | Genomes* | PAM | CRISPR Consensus† | Leaders‡ |
|---|---|---|---|---|
| Group | Mth | NGG | ATTTCAATCCCATTTTGGT | AGGGCGGATT |
| 1 | | | CTGATTTTAAC | ATGGCCAATT |
| | Lmo | WGG | ATTTACATTTCAHAATAAG | CCACTAACTT |
| | | | TARYTAAAAC | CCGCTCTATT |
| Group | Eco | CWT | CGGTTTATCCCCGCTGGCG | TCTAAACATA |
| 2 | | | CGGGGAACWC | TCTAAAAGTA |
| | Pae | CTT | CGGTTCATCCCCACRCMYG | ACTTACCGTA |
| | | | TGGGGAACAC | CCTTACCGTA |
| Group | Spy | GAA | ATTTCAATCCACTCACCCA | TGCGCCAAAT |
| 3 | | | TGAAGGGTGAGAC | |
| | Xan | GAA | GTTTCAATCCACGCGCCCG | CCCCCCTTAG |
| | | | TGAGGRCGCGAC | GCCGCCAGCA |
| Group | She | GG | TTTCTAAGCCGCCTGTGCG | AATAGCTTAT |
| 4 | | | GCGGTGAAC | TGTAGAATAA |
| | Pae | GG | TTTCTTAGCTGCCTATACG | TAGCTCCGAA |
| | | | GCAGTGAAC | TAGACCAAAA |
| | Ype | GG | TTTCTAAGCTGCCTGTGCG | GTAAGATAAT |
| | | | GCAGTGAAC | |
| Group | Sso | NGG | CTTTCAATTCTATAAGAGA | TGAGGGTTTA |
| 7 | | | TTATC | |
| | Mse | NGG | CTTTCAACTCTATAGGAGA | TGATACCTTT |
| | | | TTAAC | TGAAACTTTT |
| | | | | TGACACTCTT |
| Group | Str | NGG | GTTTTAGAGCTATGCTGTT | CTCGTAGACT |
| 10 | | | TTGAATGGTCCCAAAAC | CTCGTAGAAA |
| | Lis | NGG | GTTTTAGAGCTATGTTATT | CTCGCAGAAT |
| | | | TTGAATGCTAMCAAAAC | CTCGTAGAAT |

*Genomes are abbreviated according to the denominations of the species or genera carrying the corresponding CRISPR arrays: Mth, M. thermautotrophicus; Lmo, L. monocytogenes; Eco, E. coli, Pae, P. aeruginosa; Spy, S. pyogenes; Xan, Xanthomonas spp.; She, Shewanella spp.; Ype, Y. pestis; Sso, S. solfataricus; Mse, M. sedula; Str, Streptococcus spp.; Lis, Listeria spp.
†Sequences matching the PAM are underlined.
‡Representative CRISPR array proximal Leader sequences. Nucleotides matching the PAM are underlined.

TABLE 2
CRISPR-Associated Endonucleases
[Gene ID numbers refer to genes in the NCBI Gene Database as at September 2013; all
sequence information relating to the gene IDs below is incorporated herein by reference
for possible use in the present invention]

1. Plav_0099
CRISPR-associated endonuclease Csn1 family protein[Parvibaculum lavamentivorans DS-1]
Other Aliases: Plav _0099
Genomic context: Chromosome
Annotation: NC_009719.1 (105795 . . . 108908, complement)
ID: 5454634
2. FTN_0757
membrane protein[Francisella novicida U112]
Other Aliases: FTN_0757
Genomic context: Chromosome
Annotation: NC_008601.1 (810052 . . . 814941)
ID: 4548251
3. Cj1523c
CRISPR-associated protein[Campylobacter jejuni subsp. jejuni NCTC 11168 = ATCC 700819]
Other Aliases: Cj1523c
Genomic context: Chromosome
Annotation: NC_002163.1 (1456880 . . . 1459834, complement)
ID: 905809
4. mcrA
restriction endonuclease[Bifidobacterium longum DJO10A]
Other Aliases: BLD_1902
Genomic context: Chromosome
Annotation: NC_010816.1 (2257993 . . . 2261556)
ID: 6362834
5. MGA_0519
Csn1 family CRISPR-associated protein[Mycoplasma gallisepticum str. R(low)]
Other Aliases: MGA_0519
Genomic context: Chromosome
Annotation: NC_004829.2 (919248 . . . 923060)
ID: 1089911
6. Emin_0243
CRISPR-associated endonuclease Csn1 family protein[Elusimicrobium minutum Pei191]
Other Aliases: Emin_0243
Genomic context: Chromosome
Annotation: NC_010644.1 (261119 . . . 264706)
ID: 6263045
7. FTW_1427
CRISPR-associated large protein[Francisella tularensis subsp. tularensis WY96-3418]
Other Aliases: FTW_1427
Genomic context: Chromosome
Annotation: NC_009257.1 (1332426 . . . 1335803, complement)
ID: 4958852
8. SMA_1444
CRISPR-associated protein, Csn1 family[Streptococcus macedonicus ACA-DC 198]
Other Aliases: SMA_1444
Annotation: NC_016749.1 (1418337 . . . 1421729, complement)
ID: 11601419
9. SSUST3_1318
CRISPR-associated protein, Csn1 family[Streptococcus suis ST3]
Other Aliases: SSUST3_1318
Genomic context: Chromosome
Annotation: NC_015433.1 (1323872 . . . 1327240, complement)
ID: 10491484
10. cas5
CRISPR-associated protein, Csn1 family[Streptococcus gallolyticus UCN34]
Other Aliases: GALLO_1439
Genomic context: Chromosome
Annotation: NC_013798.1 (1511433 . . . 1514825, complement)
ID: 8776949
11. GALLO_1446
CRISPR-associated protein[Streptococcus gallolyticus UCN34]
Other Aliases: GALLO_1446
Genomic context: Chromosome
Annotation: NC_013798.1 (1518984 . . . 1523110, complement)
ID: 8776185
12. csn1
CRISPR-associated endonuclease Csn1[Bifidobacterium dentium Bd1]
Other Aliases: BDP_1254
Genomic context: Chromosome
Annotation: NC_013714.1 (1400576 . . . 1403992, complement)
ID: 8692053
13. NMO_0348
putative CRISPR-associated protein[Neisseria meningitidis alpha14]
Other Aliases: NMO_0348
Genomic context: Chromosome
Annotation: NC_013016.1 (369547 . . . 372795, complement)
ID: 8221228
14. csn1
CRISPR-Associated Protein Csn1[Streptococcus equi subsp. zooepidemicus MGCS10565]
Other Aliases: Sez_1330
Genomic context: Chromosome
Annotation: NC_011134.1 (1369339 . . . 1373385, complement)
ID: 6762114
15. csn1
CRISPR-associated endonuclease Csn1 family protein[Streptococcus gordonii str. Challis substr. CH1]
Other Aliases: SGO_1381
Genomic context: Chromosome
Annotation: NC_009785.1 (1426750 . . . 1430160, complement)
ID: 5599802
16. M28_Spy0748
cytoplasmic protein[Streptococcus pyogenes MGAS6180]
Other Aliases: M28_Spy0748
Genomic context: Chromosome
Annotation: NC_007296.1 (771231 . . . 775337)
ID: 3573516
17. SGGBAA2069_c14690
CRISPR-associated protein[Streptococcus gallolyticus subsp. gallolyticus ATCC BAA-2069]
Other Aliases: SGGBAA2069_c14690
Genomic context: Chromosome
Annotation: NC_015215.1 (1520905 . . . 1525017, complement)
ID: 10295470
18. SAR116_2544
CRISPR-associated protein, Csn1 family[Candidatus Puniceispirillum marinum IMCC1322]
Other Aliases: SAR116_2544
Genomic context: Chromosome
Annotation: NC_014010.1 (2748992 . . . 2752099)
ID: 8962895
19. TDE0327
CRISPR-associated Cas5e[Treponema denticola ATCC 35405]
Other Aliases: TDE0327
Genomic context: Chromosome
Annotation: NC_002967.9 (361021 . . . 365208)
ID: 2741543
20. csn1
CRISPR-associated protein[Streptococcus pasteurianus ATCC 43144]
Other Aliases: SGPB_1342
Genomic context: Chromosome
Annotation: NC_015600.1 (1400035 . . . 1403427, complement)
ID: 10753339
21. cas9
CRISPR-associated protein[Corynebacterium ulcerans BR-AD22]
Other Aliases: CULC22_00031
Genomic context: Chromosome
Annotation: NC_015683.1 (30419 . . . 33112, complement)
ID: 10842578
22. MGAS2096_Spy0843
putative cytoplasmic protein[Streptococcus pyogenes MGAS2096]
Other Aliases: MGAS2096_Spy0843
Genomic context: Chromosome
Annotation: NC_008023.1 (813084 . . . 817190)
ID: 4066021
23. MGAS9429_Spy0885
cytoplasmic protein[Streptococcus pyogenes MGAS9429]
Other Aliases: MGAS9429_Spy0885
Genomic context: Chromosome
Annotation: NC_008021.1 (852508 . . . 856614)
ID: 4061575
24. AZL_009000
CRISPR-associated protein, Csn1 family[Azospirillum sp. B510]
Other Aliases: AZL_009000
Genomic context: Chromosome
Annotation: NC_013854.1 (1019522 . . . 1023028, complement)
ID: 8789261
25. EUBREC_1713
contains RuvC-like nuclease and HNH-nuclease domains[Eubacterium rectale ATCC 33656]
Other Aliases: EUBREC_1713
Other Designations: CRISPR-system related protein
Genomic context: Chromosome
Annotation: NC_012781.1 (1591112 . . . 1594456)
ID: 7963668
26. Alide2_0194
CRISPR-associated protein, Csn1 family[Alicycliphilus denitrificans K601]
Other Aliases: Alide2_0194
Genomic context: Chromosome
Annotation: NC_015422.1 (218107 . . . 221196)
ID: 10481210
27. Alide_0205
crispr-associated protein, csn1 family[Alicycliphilus denitrificans BC]
Other Aliases: Alide_0205
Genomic context: Chromosome
Annotation: NC_014910.1 (228371 . . . 231460)
ID: 10102228
28. STER_1477
CRISPR-system-like protein[Streptococcus thermophilus LMD-9]
Other Aliases: STER_1477
Genomic context: Chromosome
Annotation: NC_008532.1 (1379975 . . . 1384141, complement)
ID: 4437923
29. STER_0709
CRISPR-system-like protein[Streptococcus thermophilus LMD-9]
Other Aliases: STER_0709
Genomic context: Chromosome
Annotation: NC_008532.1 (643235 . . . 646600)
ID: 4437391
30. cas9
CRISPR-associated protein[Corynebacterium diphtheriae 241]
Other Aliases: CD241_2102
Genomic context: Chromosome
Annotation: NC_016782.1 (2245769 . . . 2248399)
ID: 11674395
31. cas3
CRISPR-associated endonuclease[Corynebacterium diphtheriae 241]
Other Aliases: CD241_0034
Genomic context: Chromosome
Annotation: NC_016782.1 (35063 . . . 38317)
ID: 11672999
32. Corgl_1738
CRISPR-associated protein, Csn1 family[Coriobacterium glomerans PW2]
Other Aliases: Corgl_1738
Genomic context: Chromosome
Annotation: NC_015389.1 (2036091 . . . 2040245)
ID: 10439994
33. Fluta_3147
CRISPR-associated protein, Csn1 family[Fluviicola taffensis DSM 16823]
Other Aliases: Fluta_3147
Genomic context: Chromosome
Annotation: NC_015321.1 (3610221 . . . 3614597, complement)
ID: 10398516
34. Acav_0267
CRISPR-associated protein, Csn1 family[Acidovorax avenae subsp. avenae ATCC 19860]
Other Aliases: Acav_0267
Genomic context: Chromosome
Annotation: NC_015138.1 (295839 . . . 298976)
ID: 10305168
35. NAL212_2952
CRISPR-associated protein, Csn1 family[Nitrosomonas sp. AL212]
Other Aliases: NAL212_2952
Genomic context: Chromosome
Annotation: NC_015222.1 (2941806 . . . 2944940, complement)
ID: 10299493
36. SpiBuddy_2181
CRISPR-associated protein, Csn1 family[Sphaerochaeta globosa str. Buddy]
Other Aliases: SpiBuddy_2181
Genomic context: Chromosome
Annotation: NC_015152.1 (2367952 . . . 2371491, complement)
ID: 10292274
37. Tmz1t_2411
HNH endonuclease[Thauera sp. MZ1T]
Other Aliases: Tmz1t_2411
Genomic context: Plasmid pTha01
Annotation: NC_011667.1 (75253 . . . 76200, complement)
ID: 7094333
38. Gdia_0342
Csn1 family CRISPR-associated protein[Gluconacetobacter diazotrophicus PAI 5]
Other Aliases: Gdia_0342
Genomic context: Chromosome
Annotation: NC_011365.1 (382737 . . . 385748)
ID: 6973736
39. JJD26997_1875
CRISPR-associated Cas5e family protein[Campylobacter jejuni subsp. doylei 269.97]
Other Aliases: JJD26997_1875
Genomic context: Chromosome
Annotation: NC_009707.1 (1656109 . . . 1659063, complement)
ID: 5389688
40. Asuc_0376
CRISPR-associated endonuclease Csn1 family protein[Actinobacillus succinogenes 130Z]
Other Aliases: Asuc_0376
Genomic context: Chromosome
Annotation: NC_009655.1 (431928 . . . 435116)
ID: 5348478
41. Veis_1230
CRISPR-associated endonuclease Csn1 family protein[Verminephrobacter eiseniae EF01-2]
Other Aliases: Veis_1230
Genomic context: Chromosome
Annotation: NC_008786.1 (1365979 . . . 1369185)
ID: 4695198
42. MGAS10270_Spy0886
putative cytoplasmic protein[Streptococcus pyogenes MGAS10270]
Other Aliases: MGAS10270_Spy0886
Genomic context: Chromosome
Annotation: NC_008022.1 (844446 . . . 848552)
ID: 4063984
43. gbs0911
hypothetical protein[Streptococcus agalactiae NEM316]
Other Aliases: gbs0911
Genomic context: Chromosome
Annotation: NC_004368.1 (945801 . . . 949946)
ID: 1029893
44. NMA0631
hypothetical protein[Neisseria meningitidis Z2491]
Other Aliases: NMA0631
Genomic context: Chromosome
Annotation: NC_003116.1 (610868 . . . 614116, complement)
ID: 906626
45. Ccan_14650
hypothetical protein[Capnocytophaga canimorsus Cc5]
Other Aliases: Ccan_14650
Genomic context: Chromosome
Annotation: NC_015846.1 (1579873 . . . 1584165, complement)
ID: 10980451
46. Ipp0160
hypothetical protein[Legionella pneumophila str. Paris]
Other Aliases: Ipp0160
Genomic context: Chromosome
Annotation: NC_006368.1 (183831 . . . 187949)
ID: 3118625
47. Cbei_2080
hypothetical protein[Clostridium beijerinckii NCIMB 8052]
Other Aliases: Cbei_2080
Genomic context: Chromosome
Annotation: NC_009617.1 (2422056 . . . 2423096)
ID: 5296367
48. MMOB0330
hypothetical protein[Mycoplasma mobile 163K]
Other Aliases: MMOB0330
Genomic context: Chromosome
Annotation: NC_006908.1 (45652 . . . 49362, complement)
ID: 2807677
49. MGF_5203
Csn1 family CRISPR-associated protein[Mycoplasma gallisepticum str. F]
Other Aliases: MGF_5203
Genomic context: Chromosome
Annotation: NC_017503.1 (888602 . . . 892411)
ID: 12397088
50. MGAH_0519
Csn1 family CRISPR-associated protein[Mycoplasma gallisepticum str. R(high)]
Other Aliases: MGAH_0519
Genomic context: Chromosome
Annotation: NC_017502.1 (918476 . . . 922288)
ID: 12395725
51. Smon_1063
CRISPR-associated protein, Csn1 family[Streptobacillus moniliformis DSM 12112]
Other Aliases: Smon_1063
Genomic context: Chromosome
Annotation: NC_013515.1 (1159048 . . . 1162827, complement)
ID: 8600791
52. Spy49_0823
hypothetical protein[Streptococcus pyogenes NZ131]
Other Aliases: Spy49_0823
Genomic context: Chromosome
Annotation: NC_011375.1 (821210 . . . 825316)
ID: 6985827
53. C8J_1425
hypothetical protein[Campylobacter jejuni subsp. jejuni 81116]
Other Aliases: C8J_1425
Genomic context: Chromosome
Annotation: NC_009839.1 (1442672 . . . 1445626, complement)
ID: 5618449
54. FTF0584
hypothetical protein[Francisella tularensis subsp. tularensis FSC198]
Other Aliases: FTF0584
Genomic context: Chromosome
Annotation: NC_008245.1 (601115 . . . 604486)
ID: 4200457
55. FTT_0584
hypothetical protein[Francisella tularensis subsp. tularensis SCHU S4]
Other Aliases: FTT_0584
Genomic context: Chromosome
Annotation: NC_006570.2 (601162 . . . 604533)
ID: 3191177
56. csn1
CRISPR-associated protein[Streptococcus dysgalactiae subsp. equisimilis RE378]
Other Aliases: GGS_1116
Annotation: NC_018712.1 (1169559 . . . 1173674, complement)
ID: 13799322
57. SMUGS5_06270
CRISPR-associated protein csn1[Streptococcus mutans GS-5]
Other Aliases: SMUGS5_06270
Genomic context: Chromosome
Annotation: NC_018089.1 (1320641 . . . 1324678, complement)
ID: 13299050
58. Y1U_C1412
Csn1[Streptococcus thermophilus MN-ZLW-002]
Other Aliases: Y1U_C1412
Genomic context: Chromosome
Annotation: NC_017927.1 (1376653 . . . 1380819, complement)
ID: 12977193
59. Y1U_C0633
CRISPR-system-like protein[Streptococcus thermophilus MN-ZLW-002]
Other Aliases: Y1U_C0633
Genomic context: Chromosome
Annotation: NC_017927.1 (624274 . . . 627639)
ID: 12975630
60. SALIVA_0715
CRISPR-associated endonuclease, Csn1 family[Streptococcus salivarius JIM8777]
Other Aliases: SALIVA_0715
Annotation: NC_017595.1 (708034 . . . 711417)
ID: 12910728
61. csn1
CRISPR-associated protein csn1[Streptococcus mutans LJ23]
Other Aliases: SMULJ23_0701
Annotation: NC_017768.1 (751695 . . . 755732)
ID: 12898085
62. RIA_1455
CRISPR-associated protein, SAG0894[Riemerella anatipestifer RA-GD]
Other Aliases: RIA_1455
Genomic context: Chromosome
Annotation: NC_017569.1 (1443996 . . . 1448198)
ID: 12613647
63. STND_0658
CRISPR-associated endonuclease, Csn1 family[Streptococcus thermophilus ND03]
Other Aliases: STND_0658
Genomic context: Chromosome
Annotation: NC_017563.1 (633621 . . . 636986)
ID: 12590813
64. RA0C_1034
putative BCR[Riemerella anatipestifer ATCC 11845 = DSM 15868]
Other Aliases: RA0C_1034
Genomic context: Chromosome
Annotation: NC_017045.1 (1023494 . . . 1026931, complement)
ID: 11996006
65. Sinf_1255
CRISPR-associated protein, SAG0894 family[Streptococcus infantarius subsp. infantarius CJ18]
Other Aliases: Sinf_1255
Genomic context: Chromosome
Annotation: NC_016826.1 (1276484 . . . 1280611, complement)
ID: 11877786
66. Nitsa_1472
CRISPR-associated protein, csn1 family[Nitratifractor salsuginis DSM 16511]
Other Aliases: Nitsa_1472
Genomic context: Chromosome
Annotation: NC_014935.1 (1477331 . . . 1480729)
ID: 10148263
67. NLA_17660
hypothetical protein[Neisseria lactamica 020-06]
Other Aliases: NLA_17660
Genomic context: Chromosome
Annotation: NC_014752.1 (1890078 . . . 1893326)
ID: 10006697
68. SmuNN2025_0694
hypothetical protein[Streptococcus mutans NN2025]
Other Aliases: SmuNN2025_0694
Genomic context: Chromosome
Annotation: NC_013928.1 (737258 . . . 741295)
ID: 8834629
69. SDEG_1231
hypothetical protein[Streptococcus dysgalactiae subsp. equisimilis GGS_124]
Other Aliases: SDEG_1231
Chromosome: 1
Annotation: Chromosome 1NC_012891.1 (1176755 . . . 1180870, complement)
ID: 8111553
70. NMCC_0397
hypothetical protein[Neisseria meningitidis 053442]
Other Aliases: NMCC_0397
Genomic context: Chromosome
Annotation: NC_010120.1 (402733 . . . 405981, complement)
ID: 5796426
71. SAK_1017
hypothetical protein[Streptococcus agalactiae A909]
Other Aliases: SAK_1017
Genomic context: Chromosome
Annotation: NC_007432.1 (980303 . . . 984415)
ID: 3686185
72. M5005_Spy_0769
hypothetical protein[Streptococcus pyogenes MGAS5005]
Other Aliases: M5005_Spy_0769
Genomic context: Chromosome
Annotation: NC_007297.1 (773340 . . . 777446)
ID: 3572134
73. MS53_0582
hypothetical protein[Mycoplasma synoviae 53]
Other Aliases: MS53_0582
Genomic context: Chromosome
Annotation: NC_007294.1 (684155 . . . 688099)
ID: 3564051
74. DIP0036
hypothetical protein[Corynebacterium diphtheriae NCTC 13129]
Other Aliases: DIP0036
Genomic context: Chromosome
Annotation: NC_002935.2 (34478 . . . 37732)
ID: 2650188
75. WS1613
hypothetical protein[Wolinella succinogenes DSM 1740]
Other Aliases: WS1613
Genomic context: Chromosome
Annotation: NC_005090.1 (1525628 . . . 1529857)
ID: 2553552
76. PM1127
hypothetical protein[Pasteurella multocida subsp. multocida str. Pm70]
Other Aliases: PM1127
Genomic context: Chromosome
Annotation: NC_002663.1 (1324015 . . . 1327185, complement)
ID: 1244474
77. SPs1176
hypothetical protein[Streptococcus pyogenes SSI-1]
Other Aliases: SPs1176
Genomic context: Chromosome
Annotation: NC_004606.1 (1149610 . . . 1153716, complement)
ID: 1065374
78. SMU_1405c
hypothetical protein[Streptococcus mutans UA159]
Other Aliases: SMU_1405c, SMU.1405c
Genomic context: Chromosome
Annotation: NC_004350.2 (1330942 . . . 1334979, complement)
ID: 1028661
79. lin2744
hypothetical protein[Listeria innocua Clip11262]
Other Aliases: lin2744
Genomic context: Chromosome
Annotation: NC_003212.1 (2770707 . . . 2774711, complement)
ID: 1131597
80. csn1B
CRISPR-associated protein[Streptococcus gallolyticus subsp. gallolyticus ATCC 43143]
Other Aliases: SGGB_1441
Annotation: NC_017576.1 (1489111 . . . 1493226, complement)
ID: 12630646
81. csn1A
CRISPR-associated protein[Streptococcus gallolyticus subsp. gallolyticus ATCC 43143]
Other Aliases: SGGB_1431
Annotation: NC_017576.1 (1480439 . . . 1483831, complement)
ID: 12630636
82. cas9
CRISPR-associated protein[Corynebacterium ulcerans 809]
Other Aliases: CULC809_00033
Genomic context: Chromosome
Annotation: NC_017317.1 (30370 . . . 33063, complement)
ID: 12286148
83. GDI_2123
hypothetical protein[Gluconacetobacter diazotrophicus PAI 5]
Other Aliases: GDI_2123
Genomic context: Chromosome
Annotation: NC_010125.1 (2177083 . . . 2180235)
ID: 5792482
84. Nham_4054
hypothetical protein[Nitrobacter hamburgensis X14]
Other Aliases: Nham_4054
Genomic context: Plasmid 1
Annotation: NC_007959.1 (13284 . . . 16784, complement)
ID: 4025380
85. str0657
hypothetical protein[Streptococcus thermophilus CNRZ1066]
Other Aliases: str0657
Genomic context: Chromosome
Annotation: NC_006449.1 (619189 . . . 622575)
ID: 3165636
86. stu0657
hypothetical protein[Streptococcus thermophilus LMG 18311]
Other Aliases: stu0657
Genomic context: Chromosome
Annotation: NC_006448.1 (624007 . . . 627375)
ID: 3165000
87. SpyM3_0677
hypothetical protein[Streptococcus pyogenes MGAS315]
Other Aliases: SpyM3_0677
Genomic context: Chromosome
Annotation: NC_004070.1 (743040 . . . 747146)
ID: 1008991
88. HFMG06CAA_5227
Csn1 family CRISPR-associated protein[Mycoplasma gallisepticum CA06_2006.052-5-2P]
Other Aliases: HFMG06CAA_5227
Genomic context: Chromosome
Annotation: NC_018412.1 (895338 . . . 899147)
ID: 13464859
89. HFMG01WIA_5025
Csn1 family CRISPR-associated protein[Mycoplasma gallisepticum WI01_2001.043-13-2P]
Other Aliases: HFMG01WIA_5025
Genomic context: Chromosome
Annotation: NC_018410.1 (857648 . . . 861457)
ID: 13463863
90. HFMG01NYA_5169
Csn1 family CRISPR-associated protein[Mycoplasma gallisepticum NY01_2001.047-5-1P]
Other Aliases: HFMG01NYA_5169
Genomic context: Chromosome
Annotation: NC_018409.1 (883511 . . . 887185)
ID: 13462600
91. HFMG96NCA_5295
Csn1 family CRISPR-associated protein[Mycoplasma gallisepticum NC96_1596-4-2P]
Other Aliases: HFMG96NCA_5295
Genomic context: Chromosome
Annotation: NC_018408.1 (904664 . . . 908473)
ID: 13462279
92. HFMG95NCA_5107
Csn1 family CRISPR-associated protein[Mycoplasma gallisepticum NC95_13295-2-2P]
Other Aliases: HFMG95NCA_5107
Genomic context: Chromosome
Annotation: NC_018407.1 (871783 . . . 875592)
ID: 13461469
93. MGAS10750_Spy0921
hypothetical protein[Streptococcus pyogenes MGAS10750]
Other Aliases: MGAS10750_Spy0921
Genomic context: Chromosome
Annotation: NC_008024.1 (875719 . . . 879834)
ID: 4066656
94. XAC3262
hypothetical protein[Xanthomonas axonopodis pv. citri str. 306]
Other Aliases: XAC3262
Genomic context: Chromosome
Annotation: NC_003919.1 (3842310 . . . 3842765)
ID: 1157333
95. SSUST1_1305
CRISPR-system-like protein[Streptococcus suis ST1]
Other Aliases: SSUST1_1305
Genomic context: Chromosome
Annotation: NC_017950.1 (1293105 . . . 1297250, complement)
ID: 13017849
96. SSUD9_1467
CRISPR-associated protein, Csn1 family[Streptococcus suis D9]
Other Aliases: SSUD9_1467
Genomic context: Chromosome
Annotation: NC_017620.1 (1456318 . . . 1459686, complement)
ID: 12718289
97. BBta_3952
hypothetical protein[Bradyrhizobium sp. BTAi1]
Other Aliases: BBta_3952
Genomic context: Chromosome
Annotation: NC_009485.1 (4149455 . . . 4152649, complement)
ID: 5151538
98. CIY_03670
CRISPR-associated protein, Csn1 family[Butyrivibrio fibrisolvens 16/4]
Other Aliases: CIY_03670
Annotation: NC_021031.1 (309663 . . . 311960, complement)
ID: 15213189
99. A11Q_912
CRISPR-associated protein, Csn1 family[Bdellovibrio exovorus JSS]
Other Aliases: A11Q_912
Genomic context: Chromosome
Annotation: NC_020813.1 (904781 . . . 907864, complement)
ID: 14861475
100. MCYN0850
Csn1 family CRISPR-associated protein[Mycoplasma cynos C142]
Other Aliases: MCYN_0850
Annotation: NC_019949.1 (951497 . . . 955216, complement)
ID: 14356531
101. SaSA20_0769
CRISPR-associated protein[Streptococcus agalactiae SA20-06]
Other Aliases: SaSA20_0769
Genomic context: Chromosome
Annotation: NC_019048.1 (803597 . . . 807709)
ID: 13908026
102. csn1
CRISPR-associated protein, Csn1 family[Streptococcus pyogenes A20]
Other Aliases: A20_0810
Genomic context: Chromosome
Annotation: NC_018936.1 (772038 . . . 776144)
ID: 13864445
103. P700755_000291
CRISPR-associated protein Cas9/Csn1, subtype II[Psychroflexus torquis ATCC 700755]
Other Aliases: P700755_000291
Genomic context: Chromosome
Annotation: NC_018721.1 (312899 . . . 317428)
ID: 13804571
104. A911_07335
CRISPR-associated protein[Campylobacter jejuni subsp. jejuni PT14]
Other Aliases: A911_07335
Genomic context: Chromosome
Annotation: NC_018709.2 (1450217 . . . 1453180, complement)
ID: 13791138
105. ASU2_02495
CRISPR-associated endonuclease Csn1 family protein[Actinobacillus suis H91-0380]
Other Aliases: ASU2_02495
Genomic context: Chromosome
Annotation: NC_018690.1 (552318 . . . 555482)
ID: 13751600
106. csn1
CRISPR-associated protein[Listeria monocytogenes SLCC2540]
Other Aliases: LMOSLCC2540_2635
Annotation: NC_018586.1 (2700744 . . . 2704748, complement)
ID: 13647248
107. csn1
CRISPR-associated protein[Listeria monocytogenes SLCC5850]
Other Aliases: LMOSLCC5850_2605
Annotation: NC_018592.1 (2646023 . . . 2650027, complement)
ID: 13626042
108. csn1
CRISPR-associated protein[Listeria monocytogenes serotype 7 str. SLCC2482]
Other Aliases: LMOSLCC2482_2606
Annotation: NC_018591.1 (2665393 . . . 2669397, complement)
ID: 13605045
109. csn1
CRISPR-associated protein[Listeria monocytogenes SLCC2755]
Other Aliases: LMOSLCC2755_2607
Annotation: NC_018587.1 (2694850 . . . 2698854, complement)
ID: 13599053
110. BN148_1523c
CRISPR-associated protein[Campylobacter jejuni subsp. jejuni NCTC 11168-BN148]
Other Aliases: BN148_1523c
Annotation: NC_018521.1 (1456880 . . . 1459834, complement)
ID: 13530688
111. Belba_3201
CRISPR-associated protein Cas9/Csn1, subtype II/NMEMI[Belliella baltica DSM 15883]
Other Aliases: Belba_3201
Genomic context: Chromosome
Annotation: NC_018010.1 (3445311 . . . 3449369, complement)
ID: 13056967
112. FN3523_1121
membrane protein[Francisella cf. novicida 3523]
Other Aliases: FN3523_1121
Genomic context: Chromosome
Annotation: NC_017449.1 (1129528 . . . 1134468, complement)
ID: 12924881
113. cas9
CRISPR-associated protein Cas9/Csn1, subtype II/NMEMI[Prevotella intermedia 17]
Other Aliases: PIN17_A0201
Chromosome: II
Annotation: Chromosome IINC_017861.1 (240722 . . . 244864)
ID: 12849954
114. csn1
CRISPR-associated protein, Csn1 family[Streptococcus thermophilus JIM 8232]
Other Aliases: STH8232_0853
Annotation: NC_017581.1 (706443 . . . 709808)
ID: 12637306
115. LMOG_01918
CRISPR-associated protein[Listeria monocytogenes J0161]
Other Aliases: LMOG_01918
Genomic context: Chromosome
Annotation: NC_017545.1 (2735374 . . . 2739378, complement)
ID: 12557915
116. LMRG_02138
CRISPR-associated protein[Listeria monocytogenes 10403S]
Other Aliases: LMRG_02138
Genomic context: Chromosome
Annotation: NC_017544.1 (2641981 . . . 2645985, complement)
ID: 12554876
117. CJSA_1443
putative CRISPR-associated protein[Campylobacter jejuni subsp. jejuni IA3902]
Other Aliases: CJSA_1443
Genomic context: Chromosome
Annotation: NC_017279.1 (1454273 . . . 1457227, complement)
ID: 12250720
118. csn1
CRISPR-associated protein Csn1[Streptococcus pyogenes MGAS1882]
Other Aliases: MGAS1882_0792
Genomic context: Chromosome
Annotation: NC_017053.1 (775696 . . . 779799)
ID: 12014080
119. csn1
CRISPR-associated protein Csn1[Streptococcus pyogenes MGAS15252]
Other Aliases: MGAS15252_0796
Genomic context: Chromosome
Annotation: NC_017040.1 (778271 . . . 782374)
ID: 11991096
120. cas3
CRISPR-associated endonuclease[Corynebacterium diphtheriae HC02]
Other Aliases: CDHC02_0036
Genomic context: Chromosome
Annotation: NC_016802.1 (37125 . . . 40379)
ID: 11739116
121. cas3
CRISPR-associated endonuclease[Corynebacterium diphtheriae C7 (beta)]
Other Aliases: CDC7B_0035
Genomic context: Chromosome
Annotation: NC_016801.1 (36309 . . . 39563)
ID: 11737358
122. cas3
CRISPR-associated endonuclease[Corynebacterium diphtheriae BH8]
Other Aliases: CDBH8_0038
Genomic context: Chromosome
Annotation: NC_016800.1 (37261 . . . 40515)
ID: 11735325
123. cas3
CRISPR-associated endonuclease[Corynebacterium diphtheriae 31A]
Other Aliases: CD31A_0036
Genomic context: Chromosome
Annotation: NC_016799.1 (34597 . . . 37851)
ID: 11731168
124. cas3
CRISPR-associated endonuclease[Corynebacterium diphtheriae VA01]
Other Aliases: CDVA01_0033
Genomic context: Chromosome
Annotation: NC_016790.1 (34795 . . . 38049)
ID: 11717708
125. cas3
CRISPR-associated endonuclease[Corynebacterium diphtheriae HC01]
Other Aliases: CDHC01_0034
Genomic context: Chromosome
Annotation: NC_016786.1 (35060 . . . 38314)
ID: 11708318
126. cas9
CRISPR-associated protein[Corynebacterium diphtheriae HC01]
Other Aliases: CDHC01_2103
Genomic context: Chromosome
Annotation: NC_016786.1 (2246368 . . . 2248998)
ID: 11708126
127. PARA_18570
hypothetical protein[Haemophilus parainfluenzae T3T1]
Other Aliases: PARA_18570
Genomic context: Chromosome
Annotation: NC_015964.1 (1913335 . . . 1916493)
ID: 11115627
128. HDN1F_34120
hypothetical protein[gamma proteobacterium HdN1]
Other Aliases: HDN1F_34120
Genomic context: Chromosome
Annotation: NC_014366.1 (4143336 . . . 4146413, complement)
ID: 9702142
129. SPy_1046
hypothetical protein[Streptococcus pyogenes M1 GAS]
Other Aliases: SPy_1046
Genomic context: Chromosome
Annotation: NC_002737.1 (854757 . . . 858863)
ID: 901176
130. GBS222_0765
Hypothetical protein[Streptococcus agalactiae]
Other Aliases: GBS222_0765
Annotation: NC_021195.1 (810875 . . . 814987)
ID: 15484689
131. NE061598_03330
hypothetical protein[Francisella tularensis subsp. tularensis NE061598]
Other Aliases: NE061598_03330
Genomic context: Chromosome
Annotation: NC_017453.1 (601219 . . . 604590)
ID: 12437259
132. NMV_1993
hypothetical protein[Neisseria meningitidis 8013]
Other Aliases: NMV_1993
Annotation: NC_017501.1 (1917073 . . . 1920321)
ID: 12393700
133. csn1
hypothetical protein[Campylobacter jejuni subsp. jejuni M1]
Other Aliases: CJM1_1467
Genomic context: Chromosome
Annotation: NC_017280.1 (1433667 . . . 1436252, complement)
ID: 12249021
134. FTU_0629
hypothetical protein[Francisella tularensis subsp. tularensis TIGB03]
Other Aliases: FTU_0629
Genomic context: Chromosome
Annotation: NC_016933.1 (677092 . . . 680463)
ID: 11890131
135. NMAA_0315
hypothetical protein[Neisseria meningitidis WUE 2594]
Other Aliases: NMAA_0315
Annotation: NC_017512.1 (377010 . . . 380258, complement)
ID: 12407849
136. WS1445
hypothetical protein[Wolinella succinogenes DSM 1740]
Other Aliases: WS1445
Genomic context: Chromosome
Annotation: NC_005090.1 (1388202 . . . 1391381, complement)
ID: 2554690
137. THITE_2123823
hypothetical protein[Thielavia terrestris NRRL 8126]
Other Aliases: THITE_2123823
Chromosome: 6
Annotation: Chromosome 6NC_016462.1 (1725696 . . . 1725928)
ID: 11523019
138. XAC29_16635
hypothetical protein[Xanthomonas axonopodis Xac29-1]
Other Aliases: XAC29_16635
Genomic context: Chromosome
Annotation: NC_020800.1 (3849847 . . . 3850302)
ID: 14853997
139. M1GAS476_0830
hypothetical protein[Streptococcus pyogenes M1476]
Other Aliases: M1GAS476_0830
Chromosome: 1
Annotation: NC_020540.1 (792119 . . . 796225)
ID: 14819166
140. Piso0_000203
Piso0_000203[Millerozyma farinosa CBS 7064]
Other Aliases: GNLVRS01_PISO0A04202g
Other Designations: hypothetical protein
Chromosome: A
Annotation: NC_020226.1 (343553 . . . 343774, complement)
ID: 14528449
141. G148_0828
hypothetical protein[Riemerella anatipestifer RA-CH-2]
Other Aliases: G148_0828
Genomic context: Chromosome
Annotation: NC_020125.1 (865673 . . . 869875)
ID: 14447195
142. csn1
hypothetical protein[Streptococcus dysgalactiae subsp. equisimilis AC-2713]
Other Aliases: SDSE_1207
Annotation: NC_019042.1 (1134173 . . . 1138288, complement)
ID: 13901498
143. A964_0899
hypothetical protein[Streptococcus agalactiae GD201008-001]
Other Aliases: A964_0899
Genomic context: Chromosome
Annotation: NC_018646.1 (935164 . . . 939276)
ID: 13681619
144. FNFX1_0762
hypothetical protein[Francisella cf. novicida Fx1]
Other Aliases: FNFX1_0762
Genomic context: Chromosome
Annotation: NC_017450.1 (781484 . . . 786373)
ID: 12435564
145. FTV_0545
hypothetical protein[Francisella tularensis subsp. tularensis TI0902]
Other Aliases: FTV_0545
Genomic context: Chromosome
Annotation: NC_016937.1 (601185 . . . 604556)
ID: 11880693
146. FTL_1327
hypothetical protein[Francisella tularensis subsp. holarctica LVS]
Other Aliases: FTL_1327
Genomic context: Chromosome
Annotation: NC_007880.1 (1262508 . . . 1263689, complement)
ID: 3952607
147. FTL_1326
hypothetical protein[Francisella tularensis subsp. holarctica LVS]
Other Aliases: FTL_1326
Genomic context: Chromosome
Annotation: NC_007880.1 (1261927 . . . 1262403, complement)
ID: 3952606
63306050v1

1. A method for antibody production, comprising the steps of:
(a) providing a mouse or progeny thereof developed from the modified mouse ES cell, wherein the modified mouse ES cell has been produced by contacting a mouse ES cell with:
a Cas9 protein;
a CRISPR RNA that hybridizes to a CRISPR target sequence at the genomic locus of interest;
a tracrRNA; and
an incoming nucleic acid sequence that is flanked by:
(i)a 5′ homology arm that is homologous to a 5′ target sequence at the genomic locus of interest; and
(ii) a 3′ homolog arm that is homologous to a 3′ target sequence at the genomic locus of interest;
wherein following the contacting step, the genome of the mouse ES cell is modified to comprise a targeted genetic modification comprising a disruption of a target protein or domain thereof thereby producing a modified mouse ES cell and developing the mouse from the modified mouse ES cell;
(b) immunizing the mouse with a predetermined antigen;
(c) removing B lymphocytes from the mouse and selecting one or more B lymphocytes expressing antibodies that bind to the antigen; and
(d) isolating an antibody expressed by the B lymphocytes.

2. The method of claim 1, further comprising a step of producing the modified mouse ES cell wherein the step comprises contacting a mouse ES cell with:
a Cas9 protein;
a CRISPR RNA that hybridizes to a CRISPR target sequence at the genomic locus of interest;
a tracrRNA; and
an incoming nucleic acid sequence that is flanked by:
(i) a 5′ homology arm that is homologous to a 5′ target sequence at the genomic locus of interest; and
(ii) a 3′ homolog arm that is homologous to a 3′ target sequence at the genomic locus of interest,
wherein following the contacting step, the genome of the mouse ES cell is modified to comprise a targeted genetic modification comprising a disruption of a target protein or domain thereof thereby producing a modified mouse ES cell.

3. The method of claim 1, wherein the incoming nucleic acid is at least 10 kb in size.

4. The method of claim 1, wherein the targeted genomic modification comprises:
deletion of a region of the genomic locus of interest wherein the deletion is at least 20 kb; and/or
insertion of the insert nucleic acid at the genomic locus of interest wherein the insertion is at least 20 kb.

5. The method of claim 2, wherein prior to performing step (a), the mouse ES cell comprises a deletion of one or more mouse antibody gene segments, antibody variable regions, or antibody constant regions;
the mouse ES cell comprises an insertion of one or more human antibody gene segments, antibody variable regions, or antibody constant regions; or
the mouse ES cell comprises a deletion of one or more mouse antibody gene segments, antibody variable regions, or antibody constant regions and an insertion of one or more human antibody gene segments, antibody variable regions, and/or antibody constant regions.

6. (canceled)

7. (canceled)

8. The method of claim 1, further comprising isolating from the B lymphocytes a nucleic acid encoding the antibody.

9. The method of claim 1, further comprising:
(e) isolating from the B lymphocytes a nucleic acid encoding the antibody.

10. The method of claim 9, further comprising:
(f) exchanging the heavy chain constant region nucleotide sequence of the nucleic acid encoding the antibody with a nucleotide sequence encoding a human heavy chain constant region; humanised heavy chain constant region; or a non-naturally occurring human constant region.

11. The method of claim 9, further comprising a step of affinity maturing the variable region of the antibody and/or a step of inserting the isolated nucleic acid into an expression vector or host.

12. (canceled)

13. The method of claim 1, wherein the antibody expressed by the B lymphocytes is a IgG-type antibody.

14. The method of claim 1, wherein the method further comprises a step of immortalizing the selected B lymphocytes of progeny thereof after step (d) and/or producing hybridomas from the selected B lymphocytes of progeny thereof after step (d).

15. (canceled)

16. The method of claim 4, wherein the targeted genomic modification comprises insertion of one or more of:
a. one or more human antibody heavy chain variable domains;
b. one or more human antibody kappa light chain variable domains;
c. one or more human antibody lambda light chain variable domains; and
d. one or more human Fc receptor protein or subunit or domain thereof.

17. The method of claim 4, wherein the targeted genomic modification comprises deletion of:
one or more mouse antibody heavy chain variable domains and insertion of one or more human antibody heavy chain variable domains;
one or more mouse antibody kappa light chain variable domains and insertion of one or more human antibody kappa light chain variable domains; or
one or more mouse antibody lambda light chain variable domains and insertion of one or more human antibody lambda light chain variable domains.

18-20. (canceled)

21. The method of claim 4, wherein the targeted genomic modification comprises insertion of one or more human antibody gene segments, antibody variable regions, or antibody constant regions and/or the targeted genomic modification comprises deletion of one or more mouse antibody gene segments, antibody variable regions, or antibody constant regions and insertion of one or more human antibody gene segments, antibody variable regions, or antibody constant regions.

22. (canceled)

23. The method of claim 1, wherein the targeted genomic modification comprises a knock-out of a target protein or domain thereof.

24. The method of claim 1, wherein step (a) comprises the use of at least two CRISPR RNAs; each of which hybridizes to a CRISPR target sequence at the same genomic locus of interest.

25. The method of claim 24, wherein an indel is created at the genomic locus, wherein the indel results in a knock-out or disruption of a target protein or domain thereof.

26. A pharmaceutical composition comprising an isolated antibody produced by the method of claim 1.

27. A nucleic acid produced by the method of claim 9.

28. The method of claim 1, wherein the targeted genomic modification comprises:
deletion of a region of the genomic locus of interest wherein the deletion is at least 1 kb.


SEQUENCE LISTING (61 sequences)

SEQ ID NO: 1 - RNA, 11 nt, synthetic construct
  misc_feature 1..11: note = an example of part of gRNA before linker sequence
  source 1..11: mol_type = other RNA
ttttagagcta

SEQ ID NO: 2 - RNA, 13 nt, synthetic construct
  misc_feature 1..13: note = an example of part of gRNA following linker sequence
  source 1..13: mol_type = other RNA
tagcaagttaaaa

SEQ ID NO: 3 - skipped

SEQ ID NO: 4 - RNA, 16 nt, synthetic construct
  misc_feature 1..16: note = exemplary tracrRNA which comprises a portion of a gRNA
  variation 5: note = n = A, U, G or C
  variation 6..9: note = n = A, U, G, Cor absent
  source 1..16: mol_type = other RNA
  variation 16: note = n = A, U, G or C
tagcnnnnnttaaaan

SEQ ID NO: 5 - RNA, 26 nt, synthetic construct
  misc_feature 1..26: note = an example of a tracrRNA
  source 1..26: mol_type = other RNA
tagcaagttaaaataaggctagtccg

SEQ ID NO: 6 - RNA, 76 nt, synthetic construct
  misc_feature 1..76: note = an example of a gRNA
  source 1..76: mol_type = other RNA
gttttagagctagaaatagcaagttaaaataaggctagtccgttatcaacttgaaaaagtggcaccgagtcggtgc

SEQ ID NO: 7 - DNA, 1340 bp, synthetic construct
  misc_feature 1..1340: note = Fragment 1A (1340 bp)
  source 1..1340: mol_type = other DNA
ggtaccgggccccccctcgaggtcgacggtatcgataagcttgatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatatt

SEQ ID NO: 8 - DNA, 852 bp, synthetic construct
  misc_feature 1..852: note = Fragment 2 (852 bp)
  source 1..852: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagc

SEQ ID NO: 9 - DNA, 920 bp, synthetic construct
  misc_feature 1..920: note = Fragment 3 (920 bp)
  source 1..920: mol_type = other DNA
ggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcc

SEQ ID NO: 10 - DNA, 920 bp, synthetic construct
  misc_feature 1..920: note = Fragment 4 (920 bp)
  source 1..920: mol_type = other DNA
cgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactgg

SEQ ID NO: 11 - DNA, 920 bp, synthetic construct
  misc_feature 1..920: note = Fragment 5 (920 bp)
  source 1..920: mol_type = other DNA
actacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatc

SEQ ID NO: 12 - DNA, 789 bp, synthetic construct
  misc_feature 1..789: note = Fragment 6 (789 bp)
  source 1..789: mol_type = other DNA
agcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 13 - DNA, 535 bp, synthetic construct
  misc_feature 1..535: note = Fragment 7 (535 bp)
  source 1..535: mol_type = other DNA
ggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctatcgaattcctgcagcccgggggatccactagttctagagcggcc

SEQ ID NO: 14 - DNA, 852 bp, synthetic construct
  misc_feature 1..852: note = Fragment 2A (852 bp)
  source 1..852: mol_type = other DNA
atggactataaggaccacgacggagactacaaggatcatgatattgattacaaagacgatgacgataagatggccccaaagaagaagcggaaggtcggtatccacggagtcccagcagccgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagc

SEQ ID NO: 15-16 - skipped (2 entries)

SEQ ID NO: 17 - DNA, 122 bp, synthetic construct
  misc_feature 1..122: note = Example T7-gRNA Sequence
  variation 21..40: note = n = A, T, C or G
  source 1..122: mol_type = other DNA
ttaatacgactcactataggnnnnnnnnnnnnnnnnnnnngttttagagctagaaatagcaagttaaaataaggctagtccgttatcaacttgaaaaagtggcaccgagtcggtgctttttt

SEQ ID NO: 18 - DNA, 112 bp, synthetic construct
  misc_feature 1..112: note = Fragment 1 (111 bp)
  source 1..112: mol_type = other DNA
ggtaccgggccccccctcgaggtcgacggtatcgataagcttgataatacgactcactatagggagaatggactataaggaccacgacggagactacaaggatcatgatatt

SEQ ID NO: 19 - DNA, 20 bp, synthetic construct
  misc_feature 1..20: note = primer Cas9-F
  source 1..20: mol_type = other DNA
ttaatacgactcactatagg

SEQ ID NO: 20 - DNA, 21 bp, synthetic construct
  misc_feature 1..21: note = primer Cas9-R used for PCR amplification
  source 1..21: mol_type = other DNA
gcgagctctaggaattcttac

SEQ ID NO: 21 - DNA, 20 bp, synthetic construct
  misc_feature 1..20: note = primer CgRNA-F
  source 1..20: mol_type = other DNA
ttaatacgactcactatagg

SEQ ID NO: 22 - DNA, 24 bp, synthetic construct
  misc_feature 1..24: note = primer CgRNA-R
  source 1..24: mol_type = other DNA
aaaaaagcaccgactcggtgccac

SEQ ID NO: 23 - DNA, 3108 bp, synthetic construct
  misc_feature 1..3108: note = Sequence of Rosa26 5-prime homology arm
  source 1..3108: mol_type = other DNA
cacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtac

SEQ ID NO: 24 - DNA, 3102 bp, synthetic construct
  misc_feature 1..3102: note = Sequence of Rosa26 3-prime homology arm
  source 1..3102: mol_type = other DNA
cttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatatcccactgaacagcatcaatgggcaaaccccaggttgtttttctgtggagacaaaggtaagatatttcaatatattttcccaagctaatgagatggctcagcaaataatggtactggccattaagtctcatgacctgagcttgatcctcagggaccatgtggtacaaggagagacctaaatccttcagttggacttcaatcttctaccctcatgtccacacacaaataaatacaataaaaaacattctgcagtctgaatttctaaaggttgtttttctaaaaagaaatgttaaagtaacataggaagaaatatgtccataactgaaatacaagttttttaaatggttaagactggttttcaaaggatgtatggttaagaaaataccagggaaaatgagcttacatgtaaaaaagtgtctaaaaggccagagaaatgacccagctggcaaaggtgtctgccctaagccagacaaaaggaatttgattcacaggaagaagagacccaactctcactagttatcctctgacttccacaccatgacacagctccatggcactctcaggcccccacacatatacagatataaacagaaacctaatccaccagccttcagaagcaaagcaattggaggatttaaacaggccatggctactaatagagataactggtagtttaaaagttatggtaatgactttcatgcttctttcaactcatattgttctaaataattaatttggtttttcaaggcagggtttctctgtgtagttctggctgtcctggaactcactctgtagaccaggctggccttgaactcagatccatctgcctctggaataagggcacgtgcgtgccttttctacataacaaaacctatactataacaaaacctataccatactgtaccgttttgggaaaagacaaaaaataatgaacaaaaaaggagaaataacattccaataaagtatggaaatggtagttaaattaattacaaatgtttttcagtaaattagatgtgacttctcatactgttcatttggctataatgataccacaaagcactgggggtgaataataattccaagtcagtagggagagagacttgaaaagatgcaatgcaatcattgaagttaaacttacccatctttaatctggctcttagtcaatagagatgagatgttatttgctgctctgttcactgccagtgggttattgtccccagcaatatggtaacagtgagaccactcagtagccccctatgagacaggagtgttggttaaacatgccacaagagaaaagggaaaagtcactatggccaactctcagtaacatggcaatccgtgccattcatttccttgccagaaatgtcttccctgttcttctgcctactgaactttcacccactagaaatgtggctccaatgtcatccactatgacatcaatgtcagcgctagaagcactttgcacacctctgttgctgacttag

SEQ ID NO: 25 - DNA, 30 bp, synthetic construct
  misc_feature 1..30: note = CRISPR consensus sequence of Genome Mth and PaM NGG
  source 1..30: mol_type = other DNA
atttcaatcccattttggtctgattttaac

SEQ ID NO: 26 - DNA, 29 bp, synthetic construct
  misc_feature 1..29: note = CRISPR consensus sequence of Genome Lmo and PaM WGG
  source 1..29: mol_type = other DNA
atttacatttcahaataagtarytaaaac

SEQ ID NO: 27 - DNA, 29 bp, synthetic construct
  misc_feature 1..29: note = CRISPR consensus sequence of Genome Eco and PaM CWT
  source 1..29: mol_type = other DNA
cggtttatccccgctggcgcggggaacwc

SEQ ID NO: 28 - DNA, 29 bp, synthetic construct
  misc_feature 1..29: note = CRISPR consensus sequence of Genome Pae and PaM CTT
  source 1..29: mol_type = other DNA
cggttcatccccacrcmygtggggaacac

SEQ ID NO: 29 - DNA, 32 bp, synthetic construct
  misc_feature 1..32: note = CRISPR consensus sequence of Genome Spy and PaM GAA
  source 1..32: mol_type = other DNA
atttcaatccactcacccatgaagggtgagac

SEQ ID NO: 30 - DNA, 31 bp, synthetic construct
  misc_feature 1..31: note = CRISPR consensus sequence of Genome Xan and PaM WGG
  source 1..31: mol_type = other DNA
gtttcaatccacgcgcccgtgaggrcgcgac

SEQ ID NO: 31 - DNA, 28 bp, synthetic construct
  misc_feature 1..28: note = CRISPR consensus sequence of Genome She and PaM GG
  source 1..28: mol_type = other DNA
tttctaagccgcctgtgcggcggtgaac

SEQ ID NO: 32 - DNA, 28 bp, synthetic construct
  misc_feature 1..28: note = CRISPR consensus sequence of Genome Pae and PaM GG
  source 1..28: mol_type = other DNA
tttcttagctgcctatacggcagtgaac

SEQ ID NO: 33 - DNA, 28 bp, synthetic construct
  misc_feature 1..28: note = CRISPR consensus sequence of Genome Ype and PaM GG
  source 1..28: mol_type = other DNA
tttctaagctgcctgtgcggcagtgaac

SEQ ID NO: 34 - DNA, 24 bp, synthetic construct
  misc_feature 1..24: note = CRISPR consensus sequence of Genome Sso and PaM NGG
  source 1..24: mol_type = other DNA
ctttcaattctataagagattatc

SEQ ID NO: 35 - DNA, 24 bp, synthetic construct
  misc_feature 1..24: note = CRISPR consensus sequence of Genome Mse and PaM NGG
  source 1..24: mol_type = other DNA
ctttcaactctataggagattaac

SEQ ID NO: 36 - DNA, 36 bp, synthetic construct
  misc_feature 1..36: note = CRISPR consensus sequence of Genome Str and PaM NGG
  source 1..36: mol_type = other DNA
gttttagagctatgctgttttgaatggtcccaaaac

SEQ ID NO: 37 - DNA, 36 bp, synthetic construct
  misc_feature 1..36: note = CRISPR consensus sequence of Genome Lis and PaM NGG
  source 1..36: mol_type = other DNA
gttttagagctatgttattttgaatgctamcaaaac

SEQ ID NO: 38 - DNA, 10 bp, synthetic construct
  misc_feature 1..10: note = Leader
  source 1..10: mol_type = other DNA
agggcggatt

SEQ ID NO: 39 - DNA, 10 bp, synthetic construct
  misc_feature 1..10: note = Leader
  source 1..10: mol_type = other DNA
atggccaatt

SEQ ID NO: 40 - DNA, 10 bp, synthetic construct
  misc_feature 1..10: note = Leader
  source 1..10: mol_type = other DNA
ccactaactt

SEQ ID NO: 41 - DNA, 10 bp, synthetic construct
  misc_feature 1..10: note = Leader
  source 1..10: mol_type = other DNA
ccgctctatt

SEQ ID NO: 42 - DNA, 10 bp, synthetic construct
  misc_feature 1..10: note = Leader
  source 1..10: mol_type = other DNA
tctaaacata

SEQ ID NO: 43 - DNA, 10 bp, synthetic construct
  misc_feature 1..10: note = Leader
  source 1..10: mol_type = other DNA
tctaaaagta

SEQ ID NO: 44 - DNA, 10 bp, synthetic construct
  misc_feature 1..10: note = Leader
  source 1..10: mol_type = other DNA
acttaccgta

SEQ ID NO: 45 - DNA, 10 bp, synthetic construct
  misc_feature 1..10: note = Leader
  source 1..10: mol_type = other DNA
ccttaccgta

SEQ ID NO: 46 - DNA, 10 bp, synthetic construct
  misc_feature 1..10: note = Leader
  source 1..10: mol_type = other DNA
tgcgccaaat

SEQ ID NO: 47 - DNA, 10 bp, synthetic construct
  misc_feature 1..10: note = Leader
  source 1..10: mol_type = other DNA
ccccccttag

SEQ ID NO: 48 - DNA, 10 bp, synthetic construct
  misc_feature 1..10: note = Leader
  source 1..10: mol_type = other DNA
gccgccagca

SEQ ID NO: 49 - DNA, 10 bp, synthetic construct
  misc_feature 1..10: note = Leader
  source 1..10: mol_type = other DNA
aatagcttat

SEQ ID NO: 50 - DNA, 10 bp, synthetic construct
  misc_feature 1..10: note = Leader
  source 1..10: mol_type = other DNA
tgtagaataa

SEQ ID NO: 51 - DNA, 10 bp, synthetic construct
  misc_feature 1..10: note = Leader
  source 1..10: mol_type = other DNA
tagctccgaa

SEQ ID NO: 52 - DNA, 10 bp, synthetic construct
  misc_feature 1..10: note = Leader
  source 1..10: mol_type = other DNA
tagaccaaaa

SEQ ID NO: 53 - DNA, 10 bp, synthetic construct
  misc_feature 1..10: note = Leader
  source 1..10: mol_type = other DNA
gtaagataat

SEQ ID NO: 54 - DNA, 10 bp, synthetic construct
  misc_feature 1..10: note = Leader
  source 1..10: mol_type = other DNA
tgagggttta

SEQ ID NO: 55 - DNA, 10 bp, synthetic construct
  misc_feature 1..10: note = Leader
  source 1..10: mol_type = other DNA
tgataccttt

SEQ ID NO: 56 - DNA, 10 bp, synthetic construct
  misc_feature 1..10: note = Leader
  source 1..10: mol_type = other DNA
tgaaactttt

SEQ ID NO: 57 - DNA, 10 bp, synthetic construct
  misc_feature 1..10: note = Leader
  source 1..10: mol_type = other DNA
tgacactctt

SEQ ID NO: 58 - DNA, 10 bp, synthetic construct
  misc_feature 1..10: note = Leader
  source 1..10: mol_type = other DNA
ctcgtagact

SEQ ID NO: 59 - DNA, 10 bp, synthetic construct
  misc_feature 1..10: note = Leader
  source 1..10: mol_type = other DNA
ctcgtagaaa

SEQ ID NO: 60 - DNA, 10 bp, synthetic construct
  misc_feature 1..10: note = Leader
  source 1..10: mol_type = other DNA
ctcgcagaat

SEQ ID NO: 61 - DNA, 10 bp, synthetic construct
  misc_feature 1..10: note = Leader
  source 1..10: mol_type = other DNA
ctcgtagaat